(19) United States
(12) Patent Application Publication
(10) Pub. No.: US 2022/0213516 A1
(43) Pub. Date: Jul. 7, 2022

(54) PROCESSING ORGANIC WASTE USING A HIGHLY SPECIFIC D-LACTATE OXIDASE

(71) Applicant: TRIPLE W LTD., Netanya (IL)

(72) Inventors: Tal SHAPIRA, Even Yehuda (IL); Rotem TIDHAR, Even Yehuda (IL); Ofir AVIDAN, Lod (IL); Tsvika GREENER, Ness Ziona (IL)

(21) Appl. No.: 17/602,642

(22) PCT Filed: Apr. 7, 2020
(86) PCT No.: PCT/IL2020/050428

§ 371 Date: Oct. 8, 2021

Related U.S. Application Data
(60) Provisional application No. 62/831,758, filed on Apr. 10, 2019.

(51) Int. Cl. C12P 7/56; C12N 9/04; C12P 19/02; C12P 19/14

(52) U.S. Cl. CPC C12P 7/56; C12N 9/0006; C12P 19/14; C12P 19/02; C12Y 101/03

(57) ABSTRACT

Systems and methods for processing organic waste, particularly mixed food waste, using a D-lactate oxidase are provided. The D-lactate oxidase eliminates D-lactic acid that is present in the organic waste. The processed organic waste can be used as a substrate in industrial fermentation processes, such as production of optically-pure L-lactic acid. Further provided are systems and methods for producing L-lactic acid from organic waste, in which D-lactic acid that is endogenously found in the organic waste is eliminated using a D-lactate oxidase.

### FIELD OF THE INVENTION

The present invention relates to systems and methods for processing organic waste that utilize a D-lactate oxidase to eliminate D-lactic acid present in the waste. The processed waste can be used as a substrate in industrial fermentation processes, such as production of optically-pure L-lactic acid.

### BACKGROUND OF THE INVENTION

### Lactic Acid Fermentation

Lactic acid fermentation, namely, production of lactic acid from carbohydrate sources via microbial fermentation, has been gaining interest in recent years due to the ability to use lactic acid as a building block in the manufacture of bio-plastics. Lactic acid can be polymerized to form the biodegradable and recyclable polyester polylactic acid (PLA), which is considered a potential substitute for plastics manufactured from petroleum. PLA is used in the manufacture of various products including food packaging, disposables, fibers in the textile and hygiene products industries, and more.

Production of lactic acid by fermentation bioprocesses is preferred over chemical synthesis methods for various considerations, including environmental concerns, costs and the difficulty to generate enantiomerically pure lactic acid by chemical synthesis, which is desired for most industrial applications. The conventional fermentation process is typically based on anaerobic fermentation by lactic acid-producing microorganisms, which produce lactic acid as the major metabolic end product of carbohydrate fermentation. For production of PLA, the lactic acid generated during the fermentation is separated from the fermentation broth and purified by various processes, and the purified lactic acid is then subjected to polymerization.

Lactic acid has a chiral carbon atom and therefore exists in two enantiomeric forms, D- and L-lactic acid. In order to generate PLA that is suitable for industrial applications, the polymerization process should utilize only one enantiomer. Presence of impurities or a racemic mixture of D- and L-lactic acid results in a polymer having undesired characteristics such as low crystallinity and low melting temperature. Thus, lactic acid bacteria that produce only the L-enantiomer or only the D-enantiomer are typically used.

In currently available commercial processes, the carbohydrate source for lactic acid fermentation is typically a starch-containing renewable source such as corn and cassava root. Additional sources, such as the cellulose-rich sugarcane bagasse, have also been proposed. Typically, lactic acid bacteria can utilize reducing sugars like glucose and fructose, but do not have the ability to degrade polysaccharides like starch and cellulose. Thus, to utilize such polysaccharides the process requires adding glycolytic enzymes, typically in combination with chemical treatment, to degrade the polysaccharides and release reducing sugars.

An additional source of carbohydrates for lactic acid fermentation that has been proposed is complex organic waste, such as mixed food waste. The utilization of such organic waste as a substrate for fermentation is highly advantageous compared to lactic acid production processes which utilize source materials that are of high value as human food. Mixed food waste typically includes varied ratios of reducing sugars (glucose, fructose, lactose, etc.), starch and lignocellulosic material. However, food waste also contains endogenous D,L-lactic acid (e.g., from dairy products) that need to be removed in order to utilize the waste as a substrate for producing optically pure lactic acid (L- or D-lactic acid).

Sakai et al. (2004) Journal of Industrial Ecology, 7(3-4): 63-74 report about a recycling system for municipal food waste that combines fermentation and chemical processes to produce poly-L-lactate (PLLA). The process in Sakai et al. includes removal of endogenous D,L-lactic acid from the food waste by a Propionibacterium that consumes lactic acid as a carbon source, prior to the lactic acid fermentation step.

WO 2017/122197, assigned to the Applicant of the present invention, discloses dual action lactic-acid (LA)-utilizing bacteria genetically modified to secrete polysaccharide-degrading enzymes such as cellulases, hemicellulases, and amylases, useful for processing organic waste both to eliminate lactic acid present in the waste and degrade complex polysaccharides.

### D-lactate Oxidase

A D-lactate oxidase is an enzyme that catalyzes the oxidation of D-lactate to pyruvate and H₂O₂ using O₂ as an electron acceptor. The enzyme uses flavin adenine dinucleotide (FAD) as a co-factor for its catalytic activity.

Sheng et al. (2015) Appl Environ Microbiol., 81(12): 4098-110 studied the enzymatic basis for the growth of Gluconobacter oxydans on D-lactate. Sheng et al. identified GOX2071, a D-lactate oxidase. According to Sheng et al., GOX2071 may be useful in biosensor and biocatalysis applications.

Sheng et al. (2016) ChemCatChem, 8(16) carried out enzymatic resolution of 2-hydroxycarboxylic acids into (S)-2-hydroxycarboxylic acids using the D-lactate oxidase GOX2071 from Gluconobacter oxydans.

Li et al. (2017) ACS Sustainable Chem. Eng., 5 (4): 3456-3464 used D-lactate oxidase (D-LOX) from Gluconobacter oxydans, L-lactate oxidase (L-LOX) from Pediococcus sp., pyruvate decarboxylase from Zymomonas mobilis, and catalase from bovine liver to synthesize an in vitro enzymatic system, including different enzymatic cascades, for the production of valuable platform chemicals from racemic lactate separated from corn steep water.

CN 104745544 discloses the D-lactate oxidase GOX2071 from Gluconobacter oxydans and its application in the detection of D-lactic acid.

CN 106636022, CN 106701701, CN 106701702, CN 106701705 and CN 106754793 disclose D-lactate oxidases from various microorganisms, useful for preparing optically pure (S)-alpha-hydroxy acid esters.

Nowhere is it disclosed or suggested to use a D-lactate oxidase to eliminate D-lactic acid from organic waste, such as food waste. Additionally, nowhere is it disclosed or suggested to combine a D-lactate oxidase in industrial fermentation processes of organic waste, to eliminate D-lactic acid present in the waste.

There is a need for more cost-effective and efficient systems and methods for processing organic waste, so that the organic waste can be used as a substrate in industrial fermentation processes, such as production of optically-pure lactic acid.

### SUMMARY OF THE INVENTION

The present invention provides systems and methods for processing organic waste on a commercial scale using a D-lactate oxidase, optionally with one or more polysaccharide-degrading enzyme. The present invention further provides systems and methods for producing L-lactic acid from organic waste, in which D-lactic acid that is endogenously found in the waste is eliminated using a D-lactate oxidase.

The organic waste according to the present invention includes food waste of various types and sources, as well as agricultural waste, industrial organic waste and more. The organic waste according to the present invention comprises endogenous D, L-lactic acid, originating, for example, from natural fermentation processes. The organic waste further comprises complex polysaccharides including starch, cellulose, hemicellulose and combinations thereof.

The present invention discloses for the first time the use of a D-lactate oxidase in eliminating D-lactic acid from organic waste, such as food waste.

The present invention is based in part on the finding that the D-lactate oxidase can effectively work on a raw substrate such as organic waste, particularly mixed food waste of various types and sources, which is a viscous, highly complex substrate, the exact composition of which is unknown and varies from batch to batch, containing possible inhibitors and other factors that could negatively affect the enzyme. Hitherto described experiments with the D-lactate oxidase only tested its activity in buffer solutions.

The present invention is further based on the finding that the enzyme surprisingly shows improved activity in organic waste compared to buffer solutions, characterized by a broader range of conditions in which the enzyme is active and effectively eliminates D-lactate. More particularly, the enzyme was found to work at acidic pH values in which it was previously reported to lose its activity and stability, and at a broader range of temperatures. These findings are particularly advantageous for industrial fermentation processes utilizing organic waste as the substrate, as organic waste is typically acidic, and also typically needs to be saccharified by polysaccharide-degrading enzymes such as amylases and cellulases, which typically work at acidic pH. The enzyme is therefore useful for industrial processing and fermentation of a variety of organic wastes of different pH. In addition, the enzyme may potentially be used at different time points in the process for producing lactic acid from organic waste, and in combination with other steps such as saccharification of the waste.

The present invention is further based on the finding that the D-lactate oxidase effectively eliminates D-lactate even in the presence of a significant excess of L-lactate compared to D-lactate. This allows using the enzyme before or after fermentation, thus providing greater flexibility in its industrial utilization.

In addition, it was found that when the D-lactate oxidase is combined with a polysaccharide-degrading enzyme, such as a glucoamylase, its activity is even further improved, allowing using lower amounts of the D-lactate oxidase compared to the amounts needed when it is not combined with a polysaccharide-degrading enzyme. Also, when the D-lactate oxidase was combined with a polysaccharide-degrading enzyme such as a glucoamylase, the process required less dilution of the substrate (the organic waste). Without wishing to be bound by any particular theory or a mechanism of action, it is contemplated that the improved activity of the D-lactate oxidase in the presence of a polysaccharide-degrading enzyme stems from the reduced viscosity of the waste upon degradation of polysaccharides (e.g., starch) present in the waste into soluble sugars.

Advantageously, the D-lactate oxidase is highly specific for D-lactic acid, while L-lactic acid is substantially not consumed by the enzyme. Thus, endogenous L-lactic acid present in the organic waste is maintained in the process according to the present invention and is purified in downstream processes together with L-lactic acid produced by fermentation, thus increasing the overall yield of L-lactic acid fermentation.

In addition, the enzyme catalyzes the conversion of D-lactate to pyruvate (and H₂O₂) in a highly efficient manner, enabling near-complete or even complete elimination of D-lactic acid, which is particularly important for producing L-lactic acid that is optically pure.

As a further advantage, the use of a D-lactate oxidase to eliminate D-lactic acid from the organic waste avoids the need of conducting fermentation of a lactic acid-utilizing bacterium, as previously described, thus significantly reducing the costs involved, including operating expenditure (OPEX) as well as capital expenditure (CAPEX).

According to one aspect, the present invention provides a method for processing organic waste, the method comprising:

(i) providing an organic waste; and

(ii) digesting the organic waste with a D-lactate oxidase, to eliminate D-lactic acid present in the organic waste.

In some embodiments, the organic waste is selected from the group consisting of food waste, municipal waste, agricultural waste, plant material and combinations thereof. Each possibility represents a separate embodiment of the present invention.

In some particular embodiments, the organic waste is food waste. Food waste in accordance with the present invention encompasses food waste of plant origin. Food waste in accordance with the present invention encompasses household food waste, commercial food waste and industrial food waste. Plant material in accordance with the present invention encompasses agricultural waste and manmade products such as paper waste.

In some embodiments, the D-lactate oxidase is from Gluconobacter oxydans.

As used herein, the contacting of the D-lactate oxidase with the organic waste is carried out under conditions in which the D-lactate oxidase is active and efficiently eliminates D-lactic acid (namely, converts D-lactic acid into pyruvate and H₂O₂), for sufficient time to eliminate the D-lactic acid from the waste. As used herein, “conditions in which an enzyme is active” refers to conditions such as temperature and pH in which the enzyme effectively carries out its catalytic activity, at a level that is sufficient for a given industrial process. These conditions are also referred to herein as “suitable conditions”, and the term encompasses optimum conditions. As noted above, it was surprisingly found by the inventors of the present invention that the activity of the D-lactate oxidase in organic waste is characterized by a broader range of temperatures and pH compared to previously reported conditions for this enzyme. In some embodiments, the temperature range is 25-60° C. In some embodiments, the pH range is 5.5-8. As used herein, “elimination”, when referring to D-lactic acid from organic waste, refers to reduction to residual amounts such that there is no interference with downstream processes of producing L-lactic acid and subsequently polymerization to polylactic acid that is suitable for industrial applications. “Residual amounts” indicates less than 1% lactic acid, and even more preferably less than 0.5% D-lactic acid out of the total lactate (L+D) at the end of the fermentation (w/w). In some particular embodiments, elimination of D-lactic acid is reduction to less than 0.5% D-lactic acid out of the total lactate at the end of the fermentation (w/w).

In some embodiments, the contacting of the D-lactate oxidase with the organic waste is carried out at a temperature in the range of 25-60° C. In additional embodiments, the contacting is carried out at a temperature in the range of 37-55° C. In yet additional embodiments, the contacting is carried out at a temperature in the range of 45-55° C. In some particular embodiments, the contacting is carried out at 55° C.

In some embodiments, the contacting of the D-lactate oxidase with the organic waste is carried out at a pH in the range of 5.5-7. In additional embodiments, the contacting of the D-lactate oxidase with the organic waste is carried out at a pH in the range of 6-7. In some particular embodiments, the contacting is carried out at pH=6.

In some embodiments, the contacting of the D-lactate oxidase with the organic waste is carried out for a time period ranging from 6 to 48 hours. In additional embodiments, the contacting of the D-lactate oxidase with the organic waste is carried out for a time period ranging from 6 to 12 hours. In yet additional embodiments, the contacting of the D-lactate oxidase with the organic waste is carried out for a time period ranging from 24-48 hours. In yet additional embodiments, the contacting of the D-lactate oxidase with the organic waste is carried out for a time period ranging from 24-36 hours.

In some embodiments, the method further comprises contacting the organic waste with one or more saccharide-degrading enzyme. In some embodiments, the one or more saccharide-degrading enzyme is a polysaccharide-degrading enzyme, contacted with the organic waste to degrade polysaccharides in the organic waste to release reducing sugars (saccharify the organic waste).

In some embodiments, the one or more polysaccharide-degrading enzyme is selected from the group consisting of an amylase, a cellulase and a hemicellulase. In some particular embodiments, the one or more polysaccharide-degrading enzyme comprises a glucoamylase. In some embodiments, the method comprises contacting the organic waste with a D-lactate oxidase and a glucoamylase.

In some embodiments, the contacting with the one or more saccharide-degrading enzyme, e.g., polysaccharide-degrading enzyme, and the contacting with the D-lactate oxidase are carried out concomitantly (simultaneously). According to these embodiments, the elimination of D-lactic acid and the saccharification of the organic waste are carried out concomitantly (simultaneously). Simultaneous D-lactic acid elimination and saccharification are possible for a D-lactate oxidase and one or more polysaccharide-degrading enzyme which are active in the same pH and temperature range. Thus, in some embodiments, the D-lactate oxidase and the one or more polysaccharide-degrading enzyme are active in the same pH and temperature range. In some embodiments, the method comprises contacting the organic waste with the D-lactate oxidase and the one or more polysaccharide-degrading enzyme at a temperature and pH in which the D-lactate oxidase and the one or more polysaccharide-degrading enzyme are active. Contacting is performed for sufficient time to eliminate D-lactic acid from the waste and obtain a desired level of soluble reducing sugars.

In other embodiments, the contacting with the one or more saccharide-degrading enzyme, e.g., polysaccharide-degrading enzyme, and the contacting with the D-lactate oxidase are carried out sequentially in any order. In some particular embodiments, the contacting with the one or more saccharide-degrading enzyme, e.g., polysaccharide-degrading enzyme, is carried out prior to the contacting with the D-lactate oxidase.

In other embodiments, the D-lactate oxidase and the one or more polysaccharide-degrading enzyme are active at different pH and/or temperature range.

In some embodiments, the method comprises: (1) contacting the organic waste with the D-lactate oxidase at a first temperature, the first temperature being suitable for activity of the D-lactate oxidase, for sufficient time to eliminate D-lactic from the waste; and (2) adjusting (e.g., increasing) the temperature to a second temperature, the second temperature being suitable for activity of the one or more saccharide-degrading enzyme, e.g., polysaccharide-degrading enzymes, and contacting the organic waste with the one or more polysaccharide-degrading for sufficient time to obtain a desired level of soluble reducing sugars.

In some embodiments, the method comprises: (1) contacting the organic waste with the D-lactate oxidase at a first pH, the first pH being suitable for activity of the D-lactate oxidase, for sufficient time to eliminate D-lactic from the waste; and (2) adjusting (e.g., reducing) the pH to a second pH, the second pH being suitable for activity of the saccharide-degrading enzymes, e.g., polysaccharide-degrading enzymes, and contacting the organic waste with the one or more polysaccharide-degrading for sufficient time to obtain a desired level of soluble reducing sugars.

In some embodiments, the method comprises: (1) contacting the organic waste with the one or more saccharide-degrading enzyme, e.g., polysaccharide-degrading enzyme at a first temperature, the first temperature being suitable for activity of the one or more saccharide-degrading enzyme, for sufficient time to obtain a desired level of soluble reducing sugars; and (2) adjusting (e.g., increasing) the temperature to a second temperature, the second temperature being suitable for activity of the D-lactate oxidase, and contacting the organic waste with the D-lactate oxidase for sufficient time to eliminate D-lactic from the waste.

In some embodiments, the method comprises: (1) contacting the organic waste with the one or more saccharide-degrading enzyme, e.g., polysaccharide-degrading enzyme at a first pH, the first pH being suitable for activity of the one or more saccharide-degrading enzyme, for sufficient time to obtain a desired level of soluble reducing sugars; and (2) adjusting (e.g., increasing) the pH to a second pH, the second pH being suitable for activity of the D-lactate oxidase, and contacting the organic waste with the D-lactate oxidase for sufficient time to eliminate D-lactic from the waste.

According to another aspect, the present invention provides a system for processing organic waste, the system comprising:

(a) a source of organic waste; and

(b) a D-lactate oxidase,

wherein the D-lactate oxidase is mixed with the organic waste and eliminates D-lactic acid present in the organic waste.

In some embodiments, the D-lactate oxidase is mixed with the organic waste at a temperature in the range of 25-60° C. In additional embodiments, the D-lactate oxidase is mixed with the organic waste at a temperature in the range of 37-55° C. In yet additional embodiments, the D-lactate oxidase is mixed with the organic waste at a temperature in the range of 45-55° C. In some particular embodiments, the mixing with the D-lactate oxidase is carried out at 55° C.

In some embodiments, the D-lactate oxidase is mixed with the organic waste at a pH in the range of 5.5-7. In additional embodiments, the D-lactate oxidase is mixed with the organic waste at a pH in the range of 6-7. In some particular embodiments, the mixing with the D-lactate oxidase is carried out at pH=6.

In some embodiments, the D-lactate oxidase is mixed with the organic waste for a time period ranging from 6 to 48 hours. In additional embodiments, the D-lactate oxidase is mixed with the organic waste for a time period ranging from 6 to 12 hours. In yet additional embodiments, the D-lactate oxidase is mixed with the organic waste for a time period ranging from 24-48 hours. In yet additional embodiments, the D-lactate oxidase is mixed with the organic waste for a time period ranging from 24-36 hours.

In some embodiments, the system further comprises one or more saccharide-degrading enzyme, e.g., polysaccharide-degrading enzyme, mixed with the organic waste and degrade polysaccharides in the organic waste to release reducing sugars (saccharify the organic waste).

In some particular embodiments, the system comprises a D-lactate oxidase and a glucoamylase.

In some embodiments, the D-lactate oxidase and the one or more saccharide-degrading enzyme are mixed with the organic waste concomitantly (simultaneously). In other embodiments, the D-lactate oxidase and the one or more saccharide-degrading enzyme are mixed with the organic waste sequentially in any order.

Advantageously, as exemplified hereinbelow, effective elimination of D-lactic acid from organic waste was seen even without adding any co-factors to the medium. Thus, in some embodiments, the processing of the organic waste by the D-lactate oxidase is carried out without adding any co-factors to the medium. In some particular embodiments, the processing of the organic waste by the D-lactate oxidase is carried out without adding FAD.

According to a further aspect, the present invention provides a method for producing L-lactic acid from organic waste, the method comprising: (i) eliminating D-lactic acid originating from the organic waste using a D-lactate oxidase; and (ii) fermenting the organic waste with a lactic acid-producing microorganism that produces only L-lactate.

In some embodiments, eliminating D-lactic acid originating from the organic waste using a D-lactate oxidase is carried out prior to fermenting the organic waste with a lactic acid-producing microorganism that produces only L-lactate.

In other embodiments, eliminating D-lactic acid originating from the organic waste using a D-lactate oxidase is carried out after fermenting the organic waste with a lactic acid-producing microorganism that produces only L-lactate.

In additional embodiments, eliminating D-lactic acid originating from the organic waste using a D-lactate oxidase is carried out simultaneously with fermenting the organic waste with a lactic acid-producing microorganism that produces only L-lactate.

The organic waste used according to the present invention is typically an organic waste that was subjected to pretreatment comprising reduction in particle size and increase of surface area, and optionally also inactivation of endogenous bacteria within the waste. According to some embodiments the organic waste used in the present invention is mixed food waste which may contain organic and inorganic components such as paper or plastic packaging materials. According to these embodiments, the pretreatment may include separating the packaging material, for example by hammer mill, liquefying press, rotating auger or screw press. In some embodiments, the organic waste undergoes clarification from insoluble particles prior to contacting with the D-lactate oxidase.

The pretreatment is carried out prior to processing the waste with the D-lactate oxidase (and optionally the one or more saccharide-degrading enzyme). In some embodiments, the organic waste undergoes shredding, mincing and sterilization prior to processing with the D-lactate oxidase (and optionally the one or more saccharide-degrading enzyme). Sterilization may be carried out by methods known in the art, including for example, high pressure steam, UV radiation or sonication.

In some embodiments, the organic waste further undergoes dilution with water prior to processing with the D-lactate oxidase (and optionally the one or more saccharide-degrading enzyme). Thus, in some embodiments, the pretreatment comprises diluting the organic waste with water. In some embodiments, the organic waste is diluted with water prior to said contacting with the D-lactate oxidase. The dilution with water is typically 1:1 dilution. It is typically a dilution of 35%-40% dissolved solids to 20% to 25% solids.

According to another aspect, the present invention provides a method for producing L-lactic acid from organic waste, the method comprising:

(i) providing an organic waste;

(ii) processing the organic waste to eliminate D-lactic acid present in the waste and degrade saccharides in the waste to release soluble reducing sugars, by contacting the organic waste with a D-lactate oxidase and one or more saccharide-degrading enzyme;

(iii) fermenting the processed organic waste with a lactic acid-producing microorganism that produces only L-lactic acid, to obtain L-lactic acid; and

(iv) recovering the L-lactic acid from the fermentation broth.

Recovering lactic acid from the fermentation broth typically includes separation of the lactic acid from the fermentation broth and purification of the lactic acid. In some embodiments, the L-lactic acid is recovered from the fermentation broth as a lactate salt. “Recovering lactic acid” as used herein encompasses both recovering it as lactic acid and as a lactate salt.

In some embodiments, the step of contacting the organic waste with a D-lactate oxidase and one or more saccharide-degrading enzyme and the step of fermenting the processed organic waste with a lactic acid-producing microorganism that produces only L-lactic acid are carried out simultaneously.

In other embodiments, the step of contacting the organic waste with a D-lactate oxidase and one or more saccharide-degrading enzyme is carried out prior to the step of fermenting the processed organic waste with a lactic acid-producing microorganism that produces only L-lactic acid.

According to another aspect, the present invention provides a system for producing L-lactic acid from organic waste, comprising:

(a) a source of organic waste;

(b) a D-lactate oxidase; and

(c) a lactic acid-producing microorganism that produces only L-lactate,

wherein the D-lactate oxidase eliminates D-lactic acid originating from the organic waste, and the lactic acid-producing microorganism ferments the organic waste to produce L-lactate.

In some embodiments, the system further comprises one or more saccharide-degrading enzyme.

In some embodiments, the lactic acid-producing microorganism is mixed with the organic waste after elimination of D-lactate by the D-lactate oxidase and optionally after degradation of saccharides in the organic waste.

In other embodiments, the lactic acid-producing microorganism is mixed with the organic waste simultaneously with the D-lactate oxidase and optionally one or more saccharide-degrading enzyme, to obtain simultaneous fermentation, elimination of D-lactate and optionally saccharification.

In additional embodiments, the D-lactate oxidase is added after fermentation by the lactic acid-producing microorganism is completed.

In some embodiments, a system for producing L-lactic acid from organic waste according to the present invention comprises:

(a) a source of organic waste;

(b) a processing tank comprising a D-lactate oxidase for eliminating D-lactic acid present in the organic waste and optionally one or more saccharide-degrading enzyme for degrading saccharides present in the organic waste; and

(c) a fermentation tank comprising a lactic acid-producing microorganism that produces only L-lactic acid,

wherein the organic waste is processed in the processing tank by the D-lactate oxidase and optionally by the one or more saccharide-degrading enzyme, and the processed waste is transferred to the fermentation tank for production of L-lactic acid.

Each of the processing and fermentation tanks described herein enables controlling and modifying the temperature and pH inside the tank, and also enables mixing/agitation.

The systems of the present invention typically further include additional operating units, such as: pretreatment unit, solid/liquid separation unit, seed fermenters and washing units, as well as units connecting the various operating units, for example, for feeding the organic waste into the processing tank and subsequently to the fermentation tank. The systems may also include operating units for recovering the L-lactic acid from the fermentation broth.

According to a further aspect, the present invention provides a nucleic acid construct for expressing a D-lactate oxidase, comprising a nucleic acid sequence encoding a D-lactate oxidase as set forth in SEQ ID NO: 9, operably linked to at least one regulatory sequence comprising a promoter selected from the group consisting of: SEQ ID NO: 3, SEQ ID NO: 4 and SEQ ID NO: 5.

In some embodiments, the nucleic acid construct comprises the nucleic acid sequence encoding the D-lactate oxidase as set forth in SEQ ID NO: 9 operably linked to the promoter sequence set forth as SEQ ID NO: 3.

In additional embodiments, the nucleic acid construct comprises the nucleic acid sequence encoding the D-lactate oxidase as set forth in SEQ ID NO: 9 operably linked to the promoter sequence set forth as SEQ ID NO: 4.

In additional embodiments, the nucleic acid construct comprises the nucleic acid sequence encoding the D-lactate oxidase as set forth in SEQ ID NO: 9 operably linked to the promoter sequence set forth as SEQ ID NO: 5.

In some embodiments, the nucleic acid construct is selected from the group consisting of SEQ ID NO: 6, SEQ ID NO: 7 and SEQ ID NO: 8. Each possibility represents a separate embodiment of the present invention.

Other objects, features and advantages of the present invention will become clear from the following description, examples and drawings.

### BRIEF DESCRIPTION OF THE FIGURES

FIG. 1. Degradation of pure D-lactate dissolved in a buffer (A) and D-lactate present in organic waste (B) by a recombinant D-lactate oxidase from Gluconobacter oxydans.

FIG. 2. Degradation of D-lactate present in organic waste by different concentrations of the D-lactate oxidase.

FIG. 3. Degradation of D-lactate present in organic waste diluted 1:1 or 4:1 with water.

FIG. 4. Combination of the D-lactate oxidase with a glucoamylase.

FIG. 5. Activity of the D-lactate oxidase on organic waste at different pH (A) and different temperatures (B).

FIG. 6. Activity of the D-lactate oxidase on supernatant obtained following centrifugation of organic waste. (A) pH=6 or pH=7, 30° C.; (B) pH=6 or pH=7, 55° C.

FIG. 7. Degradation of D-lactate present in organic waste before and after lactic acid fermentation. (A) 24-hour incubation of the organic waste with different concentrations of the D-lactate oxidase; (B) change in D-lactate over time following incubation of the organic waste with 0.92 mg/ml of the D-lactate oxidase; (C) 26-hour incubation of the D-lactate oxidase with supernatant obtained following centrifugation of organic waste.

FIG. 8. Expression of the D-lactate oxidase.

FIG. 9. Degradation of D-lactate by a bacterial lysate of E. coli expressing the D-lactate oxidase under a constitutive promoter in comparison to a purified D-lactate oxidase produced as described in Example 1.

### DETAILED DESCRIPTION OF THE INVENTION

The present invention provides systems and methods for processing organic waste that utilize a D-lactate oxidase to eliminate D-lactic acid from the waste. The processed waste can then be used as a substrate in industrial fermentation processes, such as production of optically-pure L-lactic acid.

In some embodiments, a method is provided for processing organic waste to eliminate D-lactic acid present in the organic waste, the method comprising: (i) providing an organic waste; and contacting the organic waste with a D-lactate oxidase, wherein said contacting eliminates D-lactic acid present in the organic waste.

Organic waste for use with the systems and methods of the present invention includes, in some embodiments, food waste, municipal waste, agricultural waste, plant material and combinations thereof. Food waste in accordance with the present invention encompasses food waste of plant origin. Food waste in accordance with the present invention encompasses household food waste, commercial food waste and industrial food waste. The organic food waste may originate from vegetable and fruit residues, plants, cooked food, protein residues, slaughter waste, and combinations thereof. Industrial organic food waste may include factory waste such as by products, factory rejects, market returns or trimmings of inedible food portions (such as peels). Commercial organic food waste may include waste from shopping malls, restaurants, supermarkets, etc.

Plant material in accordance with the present invention encompasses agricultural waste and manmade products such as paper waste.

The organic waste according to the present invention comprises endogenous D, L-lactic acid, originating, for example, from natural fermentation processes, e.g., in dairy products. The organic waste typically further comprises complex polysaccharides including starch, cellulose, hemicellulose and combinations thereof.

The use of mixed food waste as a substrate is particularly suitable for large-scale industrial fermentation as it is heterogeneous and hence it would contain most of the required minerals and vitamins for fermentation. Further, the systems and methods disclosed herein are advantageous over currently used methods as they exhibit low fossil fuel usage, do not use valuable arable land to grow crops for feedstock, water usage is low, as is GHG emission and further, the products obtained are biodegradable.

As used herein, the term “lactic acid” refers to the hydroxycarboxylic acid with the chemical formula CH₃CH(OH)CO₂H. The terms lactic acid or lactate (lactic acid without one proton) can refer to the stereoisomers of lactic acid: L-lactic acid, D-lactic acid, or to a combination thereof.

Organic wastes to be processed according to the present invention comprise endogenous D,L-lactic acid. In order to polymerize lactic acid into polylactic acid suitable for industrial applications the lactic acid should be at least about 95% optically pure, preferably at least about 99% optically pure. Thus, in order to utilize the organic waste as a substrate for the production of optically pure L-lactic acid, it is required to selectively remove at least the unwanted D-lactic acid prior to lactic acid fermentation. Removal of at least the unwanted enantiomer from the organic waste should be performed with minimal impact on the feedstock total sugar content.

The present invention addresses this need by processing the organic waste with a D-lactate oxidase.

A “D-lactate oxidase” is an enzyme that catalyzes the oxidation of D-lactate to pyruvate and H₂O₂ using O₂ as an electron acceptor. The enzyme uses flavin adenine dinucleotide (FAD) as a co-factor for its catalytic activity. A D-lactate oxidase according to the present invention is typically a soluble D-lactate oxidase (rather than membrane-bound). Advantageously, the enzyme works directly in the organic waste to eliminate the D-lactic acid. In some embodiments, the D-lactate oxidase is from Gluconobacter sp. In some embodiments, the D-lactate oxidase is from Gluconobacter oxydans (see, for example, GenBank accession number: AAW61807). In some embodiments, the D-lactate oxidase comprises an amino acid sequence with at least 75% sequence identity with the sequence set forth in SEQ ID NO: 1, for example at least 80%, at least 85%, at least 90%, at least 95%, at least 99% sequence identity with the sequence set forth in SEQ ID NO: 1. Each possibility represents a separate embodiment of the present invention. In some embodiments, the D-lactate oxidase comprises an amino acid sequence as set forth in SEQ ID NO: 1. In some embodiments, the D-lactate oxidase consists of an amino acid sequence with at least 75% sequence identity with the sequence as set forth in SEQ ID NO: 1, for example at least 80%, at least 85%, at least 90%, at least 95%, at least 99% sequence identity with the sequence set forth in SEQ ID NO: 1. Each possibility represents a separate embodiment of the present invention. In some embodiments, the D-lactate oxidase consists of the amino acid sequence set forth in SEQ ID NO: 1.

SEQ ID NO: 1:
MPEPVMTASSASAPDRLQAVLKALQPVMGERISTAPSVREEHSHGEAMNA
SNLPEAVVFAESTQDVATVLRHCHEWRVPVVAFGAGTSVEGHVVPPEQAI
SLDLSRMTGIVDLNAEDLDCRVQAGITRQTLNVEIRDTGLFFPVDPGGEA
TIGGMCATRASGTAAVRYGTMKENVLGLTVVLATGEIIRTGGRVRKSSTG
YDLTSLFVGSEGTLGIITEVQLRLHGRPDSVSAAICQFESLHDAIQTAME
IIQCGIPITRVELMDSVQMAASIQYSGLNEYQPLTTLFFEFTGSPAAVRE
QVETTEAIASGNNGLGFAWAESPEDRTRLWKARHDAYWAAKAIVPDARVI
STDCIVPISRLGELIEGVHRDIEASGLRAPLLGHVGDGNFHTLIITDDTP
EGHQQALDLDRKIVARALSLNGSCSGEHGVGMGKLEFLETEHGPGSLSVM
RALKNTMDPHHILNPGKLLPPGAVYTG

The nucleic acid sequence from Gluconobacter oxydans encoding the D-lactate oxidase is set forth in SEQ ID NO: 2.

SEQ ID NO: 2:
ATGCCGGAACCAGTCATGACCGCCTCTTCCGCCTCCGCTCCGGACCGCC
TTCAGGCCGTTCTCAAAGCCCTCCAGCCCGTCATGGGTGAGCGGATCAG
CACGGCACCCTCCGTTCGCGAAGAGCACAGCCACGGCGAGGCCATGAAT
GCCTCCAACCTGCCCGAGGCGGTGGTGTTTGCTGAAAGTACTCAGGATG
TCGCAACCGTCCTGCGGCACTGCCATGAATGGCGCGTTCCGGTCGTGGC
GTTCGGCGCTGGCACGTCCGTCGAAGGTCATGTCGTGCCGCCCGAACAG
GCCATCAGCCTCGATCTGTCACGCATGACGGGGATCGTGGACCTGAACG
CCGAGGATCTGGATTGCCGGGTCCAAGCCGGCATCACGCGCCAGACGCT
GAATGTTGAAATCCGCGATACGGGCCTGTTCTTTCCGGTCGATCCGGGT
GGGGAAGCTACGATCGGCGGTATGTGCGCCACCCGCGCCTCGGGCACGG
CCGCCGTACGCTACGGCACGATGAAAGAAAATGTGCTGGGCCTGACGGT
TGTTCTCGCGACCGGCGAAATCATCCGCACAGGTGGCCGCGTCCGCAAA
TCGTCCACCGGCTATGACCTGACATCGCTGTTCGTCGGCTCGGAAGGTA
CGCTCGGGATCATCACCGAAGTCCAGCTCCGTCTGCATGGGCGTCCAGA
CAGTGTTTCGGCCGCGATCTGCCAATTCGAAAGCCTGCATGACGCCATC
CAGACTGCCATGGAAATCATCCAGTGCGGCATCCCCATCACCCGCGTGG
AACTGATGGACAGCGTGCAGATGGCAGCTTCCATCCAGTATTCCGGCCT
GAACGAATATCAGCCGCTGACCACGCTGTTTTTCGAGTTCACAGGCTCG
CCCGCAGCGGTACGCGAGCAGGTCGAGACGACCGAAGCCATTGCGTCCG
GCAATAACGGGCTTGGCTTTGCCTGGGCCGAAAGTCCCGAAGACCGCAC
CCGCCTCTGGAAAGCGCGGCATGACGCCTACTGGGCGGCCAAGGCCATC
GTTCCGGATGCGCGCGTCATTTCCACAGACTGCATCGTCCCGATTTCCC
GTCTGGGCGAACTGATCGAGGGCGTGCATCGCGATATCGAGGCCTCCGG
CCTGCGCGCGCCCCTTCTGGGCCATGTGGGGGACGGCAATTTCCATACG
CTCATCATCACGGACGACACCCCCGAAGGGCATCAGCAGGCCCTCGATC
TGGACCGGAAGATCGTAGCCCGCGCCCTTTCGCTGAACGGGTCGTGCAG
CGGGGAACATGGTGTCGGCATGGGCAAGCTGGAGTTTCTGGAAACCGAG
CATGGGCCTGGAAGCCTCAGCGTGATGCGCGCCCTGAAGAACACGATGG
ATCCGCACCATATCCTCAATCCCGGCAAGCTCCTTCCGCCCGGTGCTGT
TTACACGGGCTGA

A D-lactate oxidase according to the present invention may be obtained by recombinant production in a host cell, for example in bacteria or fungi.

Exemplary production systems of a D-lactate oxidase:

1) E. coli production: the enzyme is expressed as a non-secreted protein. The host cells are disrupted and the cell debris are removed, e.g., by filtration (the biomass may be recycled in the process as nutrients for lactic acid production). The enzyme is purified or alternatively the crude supernatant is used as is.

2) Fungal or yeast production (e.g., production in Aspergillus niger, Myceliophthora thermophila or Pichia pastoris, each possibility represents a separate embodiment): the enzyme is expressed as a secreted protein. The host cells are removed, e.g., by filtration (and optionally recycled, as above). The enzyme is purified or alternatively the crude enzyme supernatant is used as is.

A “D-lactate oxidase” as used herein encompasses a purified enzyme and a crude supernatant of a microorganism recombinantly expressing the D-lactate oxidase, e.g., a crude supernatant of E. coli, A. niger, M. thermophila or Pichia pastoris expressing the D-lactate oxidase. A crude supernatant of a bacterium expressing the D-lactate oxidase as a non-secreted protein is also referred to herein as a “lysate” of the bacterium, or “bacterial lysate”. In some embodiments, the present invention provides a nucleic acid construct for expressing a D-lactate oxidase, particularly for expressing a D-lactate oxidase in E. coli, comprising a nucleic acid sequence encoding a D-lactate oxidase as set forth in SEQ ID NO: 9, operably linked to at least one regulatory sequence comprising a promoter selected from the group consisting of: SEQ ID NO: 3, SEQ ID NO: 4 and SEQ ID NO: 5.

As used herein, the term “nucleic acid construct” refers to an artificially assembled or isolated nucleic acid molecule which includes a nucleic acid sequence encoding a protein of interest and which is assembled such that the protein of interest is expressed in a target host cell. The nucleic acid construct comprises appropriate regulatory sequences operably linked to the nucleic acid sequence encoding the protein of interest. The nucleic acid construct may further include a nucleic acid sequence encoding a purification tag/peptide/protein.

The terms “nucleic acid sequence” and “polynucleotide” are used herein to refer to polymers of deoxyribonucleotides (DNA), ribonucleotides (RNA) and modified forms thereof in the form of a separate fragment or as a component of a larger construct. A nucleic acid sequence may be a coding sequence, i.e., a sequence that encodes for an end product in the cell, such as a protein. A nucleic acid sequence may also be a regulatory sequence, such as, for example, a promoter.

The term “regulatory sequences” refer to DNA sequences which control the expression (transcription) of coding sequences, such as promoters.

The term “promoter” is directed to a regulatory DNA sequence which controls or directs the transcription of another DNA sequence in vivo or in vitro. Usually, the promoter is located in the 5′ region (that is, precedes, located upstream) of the transcribed sequence. Promoters may be derived in their entirety from a native source, or be composed of different elements derived from different promoters found in nature, or even comprise synthetic nucleotide segments. Promoters can be constitutive (i.e. promoter activation is not regulated by an inducing agent and hence rate of transcription is constant), or inducible (i.e., promoter activation is regulated by an inducing agent). In most cases the exact boundaries of regulatory sequences have not been completely defined, and in some cases cannot be completely defined, and thus DNA sequences of some variation may have identical promoter activity.

The term “operably linked” means that a selected nucleic acid sequence is in proximity with a regulatory element (e.g. promoter) to allow the regulatory element to regulate expression of the selected nucleic acid sequence.

In some embodiments, the organic waste is mixed in a tank (e.g., a reactor) with the D-lactate oxidase prior to lactic acid fermentation under conditions optimal (or otherwise suitable) for the enzyme activity. The organic waste that is mixed with the enzyme is typically a pretreated organic waste that was subjected to pretreatment comprising reduction of particle size and optionally sterilization, dilution and separation of packaging material. In some embodiments, the D-lactate oxidase is incubated with the organic waste prior to the fermentation for sufficient time to eliminate D-lactic acid from the waste. In other embodiments, the D-lactate oxidase is incubated with the organic waste prior to lactic acid fermentation for a time sufficient to obtain partial degradation of D-lactate present in the waste, and the degradation of D-lactate continues during fermentation until the D-lactate is eliminated. In some embodiments, the organic waste is further mixed with one or more saccharide-degrading enzyme prior to lactic acid fermentation, either simultaneously with the D-lactate oxidase or sequentially in any order. Each possibility represents a separate embodiment of the present invention.

In additional embodiments, the organic waste (typically pretreated organic waste as described above) is mixed in a tank (e.g. a reactor) with the D-lactate oxidase and an L-lactic acid-producing microorganism, to obtain simultaneous D-lactic acid elimination and L-lactic acid fermentation.

In additional embodiments, the organic waste (typically pretreated organic waste as described above) is mixed in a tank (e.g. a reactor) with the D-lactate oxidase, one or more saccharide-degrading enzyme and an L-lactic acid-producing microorganism, to obtain simultaneous D-lactic acid elimination, saccharification and L-lactic acid fermentation.

In additional embodiments, elimination of D-lactic acid is carried out after fermentation is completed. In some embodiments, the fermentation broth is contacted with the D-lactate oxidase following fermentation, to eliminate D-lactic acid from the fermentation broth. In some embodiments, following fermentation, the pH of the fermentation broth is adjusted to a pH optimal (or otherwise suitable) for the D-lactate oxidase, and the D-lactate oxidase is contacted with the fermentation broth to eliminate D-lactate from the fermentation broth. In some embodiments, the degradation reaction by the D-lactate oxidase is stopped after 5-15 hours, for example after 5-10 hours, including any value within the range. The degradation reaction may be stopped, for example, by changing the pH or temperature to values in which the enzyme is inactive, for example, pH 4.5 and/or temperature of at least 65° C.

“Saccharide-degrading enzymes” as used herein refers to hydrolytic enzymes (or enzymatically-active portions thereof) that catalyze the breakdown of saccharides, including bi-saccharides (di-saccharides), oligosaccharides, polysaccharides and glycoconjugates. Saccharide-degrading enzymes may be selected from the group consisting of glycoside hydrolases, polysaccharide lyases and carbohydrate esterases. Each possibility represents a separate embodiment of the present invention. The saccharide-degrading enzymes for use with the present invention are selected from those that are active towards saccharides (such as polysaccharides) found in organic wastes, e.g. food waste. In some embodiments, the saccharide-degrading enzymes may be modified enzymes (i.e., enzymes that have been modified and are different from their corresponding wild-type enzymes). In some embodiments, the modification may include one or more mutations that result in improved property(ies) of the enzymes, such as improved activity and/or stability. In some embodiments, the saccharide-degrading enzymes are wild type (WT) enzymes.

The broad group of saccharide-degrading enzymes is divided into enzyme classes and further into enzyme families according to a standard classification system (Cantarel et al. 2009 Nucleic Acids Res 37: D233-238). An informative and updated classification of such enzymes is available on the Carbohydrate-Active Enzymes (CAZy) server (www.cazy.org).

In some embodiments, saccharide-degrading enzyme(s) used in the present invention are polysaccharide-degrading enzyme(s). In some embodiments, the one or more polysaccharide-degrading enzyme is a glycoside hydrolase. In some embodiments, the one or more polysaccharide-degrading enzyme is a glycoside hydrolase selected from the group consisting of an amylase, a cellulase and a hemicellulase. Each possibility represents a separate embodiment of the present invention. In some particular embodiments, the one or more polysaccharide-degrading enzyme is a glucoamylase.

In some embodiments, the saccharide-degrading enzyme(s) used in the present invention are disaccharide-degrading enzyme(s). In some embodiments, a disaccharide-degrading enzyme for use with the present invention is selected from a lactase and an invertase. Each possibility represents a separate embodiment of the present invention.

In some embodiments, a single saccharide-degrading enzyme is used. In other embodiments, a plurality of saccharide-degrading enzymes are used.

Saccharide-degrading enzymes for use in accordance with the present invention may be bacterial enzymes. In some embodiments, the one or more saccharide-degrading enzyme is from a thermophilic bacterium. The term “thermophilic bacterium” as used herein indicates a bacterium that thrives at temperatures higher than about 45° C., preferably above 50° C. Typically, thermophilic bacteria according to the present invention have optimum growth temperature of between about 45° C. to about 75° C., preferably about 50-70° C. In some embodiments, the thermophilic bacterium is selected from the group consisting of Clostridium sp., Paenibacillus sp., Thermobifida fusca, Bacillus sp., Geobacillus sp., Chromohalobacter sp. and Rhodothermus marinus. Each possibility represents a separate embodiment of the present invention. Non-limiting examples of thermophilic bacterial sources for saccharide-degrading enzymes include: Cellulases and hemicellulases-Clostridium sp. (e.g. Clostridium thermocellum), Paenibacillus sp., Thermobifida fusca; Amylases-Bacillus sp. (e.g. Bacillus stearothermophilus), Geobacillus sp. (e.g. Geobacillus thermoleovorans), Chromohalobacter sp., Rhodothermus marinus. Each possibility is a separate embodiment.

In other embodiments, the one or more saccharide-degrading enzyme is a from mesophilic bacterium. The term “mesophilic bacterium” as used herein indicates a bacterium that thrives at temperatures between about 20° C. and 45° C. In some embodiments, the mesophilic bacterium is selected from the group consisting of Klebsiella sp., Cohnella sp., Streptomyces sp., Acetivibrio cellulolyticus, Ruminococcus albus; Bacillus sp. and Lactobacillus fermentum. Each possibility represents a separate embodiment of the present invention. Non-limiting examples of mesophilic bacterial sources for saccharide-degrading enzymes include: Cellulases and hemicellulases-Klebsiella sp. (e.g. Klebsiella pneumonia), Cohnella sp., Streptomyces sp., Acetivibrio cellulolyticus, Ruminococcus albus; Amylases-Bacillus sp. (e.g. Bacillus amyloliquefaciens, Bacillus subtilis, Bacillus licheniformis), Lactobacillus fermentum. Each possibility is a separate embodiment. A person of skill in the art understands that some mesophilic bacteria (e.g. several Bacillus sp.) produce thermostable enzymes.

In additional embodiments, the one or more saccharide-degrading enzyme is a fungal enzyme. In some embodiments, the fungi are selected from the group consisting of Trichoderma reesei, Humicola insolens, Fusarium oxysporum, Aspergillus oryzae, Penicillium fellutanum and Thermomyces lanuginosus. Each possibility represents a separate embodiment of the present invention. Non-limiting examples of fungal sources for saccharide-degrading enzymes include: Cellulases and hemicellulases-Trichoderma reesei, Humicola insolens, Fusarium oxysporum; Amylases-Aspergillus oryzae, Penicillium fellutanum, Thermornyces lanuginosus. Each possibility is a separate embodiment.

The one or more saccharide-degrading enzyme is typically exogenously added and mixed with the organic waste, either simultaneously or sequentially, with the D-lactate oxidase. Alternatively, one or more saccharide-degrading enzyme may be expressed and secreted from the lactic acid-producing microorganism that is used in the lactic acid fermentation step.

Saccharide-degrading enzymes for use according to the present invention are commercially available, and/or may be produced recombinantly.

The D-lactate oxidase and optionally the one or more saccharide-degrading enzyme may be produced by expressing a polynucleotide molecule encoding the desired protein in a host cell, for example, in a microorganism cell transformed with the polynucleotide molecule. A DNA sequence encoding the protein may be isolated from a microorganism producing it. For example, a DNA sequence encoding the protein may be amplified from genomic DNA of the microorganism by polymerase chain reaction (PCR). The genomic DNA may be extracted from the microorganism cell prior to the amplification. Following amplification, the amplification products may be isolated and cloned into a cloning vector or directly into an expression vector that is appropriate for its expression in the host cell that was selected. Upon isolation and cloning of the polynucleotide encoding the protein, mutation(s) may be introduced by modification at one or more base pairs.

An alternative method to obtaining a polynucleotide encoding a desired protein is chemical synthesis of polynucleotides, using methods such as phosphoramidite DNA synthesis. The use of synthetic genes allows production of an artificial gene which comprises an optimized sequence of nucleotides to be expressed in desired species (for example, E. coli). The polynucleotide thus produced may then be subjected to further manipulations, including one or more of purification, annealing, ligation, amplification, digestion by restriction endonucleases and cloning into appropriate vectors. The polynucleotide may be ligated either initially into a cloning vector, or directly into an expression vector that is appropriate for its expression in the host cell that was selected.

As is readily apparent to those of skill in the art, one or more codon used in the polynucleotide for encoding particular amino acid(s) may be modified in accordance with the known and favored codon usage of the host cell which was selected for expressing the polynucleotide.

A polynucleotide according to the present invention may include non-coding sequences, including for example, non-coding 5′ and 3′ sequences, such as transcribed, non-translated sequences, termination signals, ribosome binding sites, sequences that stabilize mRNA, introns and polyadenylation signals. The polynucleotide may also include sequences that encode tags or markers fused to the protein of interest that facilitate purification, such as a His-tag. It may also be convenient to include a proteolytic cleavage site between the tag portion and the protein of interest to allow removal of the tag, such as a thrombin cleavage site.

A polynucleotide according to the present invention may be incorporated into a wide variety of expression vectors, which may be transformed into in a wide variety of host cells. A host cell according to the present invention may be prokaryotic (e.g., the bacterium Escherichia coli) or eukaryotic (e.g., the fungus Pichia pastoris). Introduction of a polynucleotide into the host cell can be effected by well known methods, such as chemical transformation (e.g. calcium chloride treatment), electroporation, conjugation, transduction, calcium phosphate transfection, DEAE-dextran mediated transfection, transvection, microinjection, cationic lipid-mediated transfection, scrape loading, ballistic introduction and infection.

Selection of a host cell transformed with the desired vector may be accomplished using standard selection protocols involving growth in a selection medium which is toxic to non-transformed cells. For example, E. coli may be grown in a medium containing an antibiotic selection agent; cells transformed with the expression vector which further provides an antibiotic resistance gene, will grow in the selection medium.

Upon transformation of a suitable host cell, and propagation under conditions appropriate for protein expression, the protein of interest may be identified in cell extracts of the transformed cells. Transformed hosts expressing the protein of interest may be identified by analyzing the proteins expressed by the host using SDS-PAGE and comparing the gel to an SDS-PAGE gel obtained from the host which was transformed with the same vector but not containing a nucleic acid sequence encoding the protein of interest. The protein of interest can also be identified by other known methods such as immunoblot analysis using suitable antibodies, dot blotting of total cell extracts, limited proteolysis, mass spectrometry analysis, and combinations thereof.

The protein of interest may be isolated and purified by conventional methods, including ammonium sulfate or ethanol precipitation, acid extraction, salt fractionation, ion exchange chromatography, hydrophobic interaction chromatography, gel permeation chromatography, affinity chromatography, and combinations thereof. The isolated protein of interest may be analyzed for its various properties, for example specific activity.

Conditions for carrying out the aforementioned procedures as well as other useful methods are readily determined by those of ordinary skill in the art

The proteins according to the present invention may be produced and/or used without their start codon (methionine or valine) and/or without their leader (signal) peptide to favor production and purification of recombinant proteins. As referred to herein, the terms “nucleic acid”, “nucleic acid sequence”, “polynucleotide”, “nucleotide” and “nucleotide sequence” may interchangeably be used. The terms are directed to polymers of deoxyribonucleotides (DNA), ribonucleotides (RNA), and modified forms thereof in the form of a separate fragment or as a component of a larger construct. The terms further include oligonucleotides composed of naturally occurring bases, sugars, and covalent internucleoside linkages, as well as oligonucleotides having non-naturally occurring portions, which function similarly to respective naturally occurring portions. A DNA may include, for example, genomic DNA, plasmid DNA, recombinant DNA or complementary DNA (cDNA). An RNA may include, for example, messenger RNA (mRNA), ribosomal RNA (rRNA) or transfer RNA (tRNA). In some embodiments, the nucleic acid sequence may be a coding sequence (i.e., a sequence that can encode for an end product in the cell, such as, a protein or a peptide). In some embodiments, the nucleic acid sequence may be a regulatory sequence (such as, for example, a promoter).

The terms “polypeptide,” “peptide” and “protein” are used interchangeably herein to refer to a polymer of amino acid residues. The terms apply to amino acid polymers composed of amino acids that occur in nature and also to amino acid polymers in which one or more amino acid residue is an artificial chemical analogue of a corresponding naturally occurring amino acid. The term “amino acid sequence” relates to a sequence composed of any one of naturally occurring amino acids, amino acids that have been chemically modified, or synthetic amino acids. The term relates to peptides and proteins, as well as fragments, analogs, derivatives and combinations of peptides and proteins.

In some embodiments, a sequence (such as, nucleic acid sequence and amino acid sequence) that is “homologous” to a reference sequence refers herein to percent identity between the sequences. The percent identity may be at least 80%, at least 85%, at least 90%, at least 95%, at least 98% or at least 99%. The percent identity can be distributed over the entire length of the sequences. Accordingly, homologous sequences can include, for example, variations related to mutations (such as, truncations, substitutions, deletions and/or additions of at least one amino acid or at least one nucleotide). For enzymes according to the present invention, it is understood that a homolog at least retains the properties and activity of the wild-type enzyme, to the extent that the homolog is useful for similar purposes as the wild-type.

The processing according to the present invention is typically carried out in a vessel such as a reactor or another suitable operating unit that enables mixing the organic waste with enzyme(s) and controlling parameters such as temperature and pH.

In some embodiments, the organic waste undergoes pretreatment comprising particle size reduction and optionally sterilization prior to contacting with the D-lactate oxidase (and optionally with one or more saccharide-degrading enzyme). The pretreatment may include, for example, shredding, grinding and sterilization, e.g., by pressurized steam. Pretreatment may also include mincing with an equal amount of water using a waste mincer, such as, e.g., an extruder, sonicator, shredder or blender.

In some embodiments, following the above pretreatment, the amount of D-lactate and/or soluble reducing sugars is determined. Such determination may be useful for downstream fermentation processes utilizing the soluble reducing sugars, enabling control of the concentration of fed sugars.

In some embodiments, following processing of the organic waste to eliminate D-lactic acid and optionally degrade saccharides into soluble reducing sugars, the processed organic waste is subjected to fermentation by a lactic acid-producing microorganism.

In some embodiments, following the processing according to the present invention the processed waste is pumped directly into a fermenter for lactic acid production. In other embodiments, following the processing according to the present invention the processed waste is subjected to additional processing prior to fermentation, such as solid/liquid separation, to remove insoluble particles prior to fermentation.

Organic wastes typically include nitrogen sources and other nutrients needed for the lactic acid-producing microorganism, but such nutrients may also be supplied separately if needed.

Typically, the fermenting step is carried out under anaerobic or microaerophilic conditions. The fermenting step is typically selected from the group consisting of batch, fed-batch, continuous and semi-continuous fermentation. Each possibility represents a separate embodiment of the present invention.

The reducing sugars in the organic waste (those originally found in the waste and those released by the action of one or more saccharide-degrading enzyme) may be fermented to lactic acid by a lactic acid-producing microorganism. To generate only L-lactic acid, the lactic acid-producing microorganism that is used is a microorganism that produces only the L-lactic acid enantiomer. The microorganism may produce only L-lactic acid naturally, or may be genetically modified to produce only L-lactic acid, for example by knocking out one or more enzymes involved in the synthesis of the undesired D-lactic acid enantiomer.

In some embodiments, following processing, the processed organic waste is transferred to a separate reactor (e.g., fermenter) for the lactic acid fermentation.

In other embodiments, the lactic acid fermentation may be carried out in the same reactor where processing of the organic waste by D-lactate oxidase and optionally one or more saccharide-degrading enzyme was carried out.

LA-producing microorganisms include various bacteria (including for example Lactobacillus species and Bacillus species) and fungi. Typically, the fermenting step is carried out under anaerobic or microaerophilic conditions, using batch, fed-batch, continuous or semi-continuous fermentation.

In batch fermentation, the carbon substrates and other components are loaded into the reactor, and, when the fermentation is completed, the product is collected. Except for neutralizing agents for pH control, other ingredients are not added to the reaction before it is completed. The inoculum size is typically about 5-10% of the liquid volume in the reactor. The fermentation is kept at substantially constant temperature and pH, where the pH is maintained by adding a suitable neutralizing agent, such as an alkali, a carbonate or ammonia.

In fed-batch fermentation, the substrate is fed continuously or sequentially to the reactor without the removal of fermentation broth (i.e., the product(s) remain in the reactor until the end of the run). Common feeding methods include intermittent, constant, pulse-feeding and exponential feeding.

In continuous fermentation, the substrate is added to the reactor continuously at a fixed rate, and the fermentation products are taken out continuously.

In semi-continuous processes, a portion of the culture is withdrawn at intervals and fresh medium is added to the system. Repeated fed-batch culture, which can be maintained indefinitely, is another name of the semi-continuous process.

During fermentation, bases such as ammonium-, sodium-, potassium-, magnesium- or calcium hydroxide may be added to maintain the pH, by neutralizing the lactic acid, with the formation of lactate salts.

Lactic acid fermentation is typically carried out for about 1-3 days or any amount therebetween, for example, 1-2 days.

After fermentation is completed, the broth containing lactic acid (or a lactate salt) may be clarified by centrifugation or passed through a filter press to separate solid residue from the fermented liquid. The filtrate may be concentrated, e.g. using a rotary vacuum evaporator.

Separation and purification of lactic acid from the broth may be carried out by methods such as distillation, extraction, electrodialysis, adsorption, ion-exchange, crystallization and combinations of these methods. Several methods are reviewed, for example, in Ghaffar et al. (2014), supra; and López-Garzón et al. (2014) Biotechnol Adv., 32(5):873-904). Alternatively, recovery and conversion of lactic acid to lactide in a single step may be used (Dusselier et al. (2015) Science, 349(6243):78-80).

In some embodiments, the composition of the organic waste in terms of reducing sugars and saccharides may be determined prior to processing using methods known in the art, including for example enzymatic assays (colorimetric, fluorometric) with glucose oxidase, hexokinase or phosphoglucose isomerase for fructose determination. Alternatively, HPLC and/or reducing sugars continuous sensors can be utilized. Total sugar analysis can be performed, for example, by phenol-sulfuric assay. The composition of the organic waste, for example percentage of at least one of starch, cellulose and hemicelluloses, may be used for selecting the one or more polysaccharide-degrading enzyme to be contacted with the organic waste.

The content of D-lactic acid following processing with the D-lactate oxidase may be measured, for example, using a specific D-lactate measuring kit (Sigma).

### PLA Recycling Process

PLA resins are typically compounded with other materials to generate desired properties. PLLA and PDLA are typically mixed to form copolymers of PLLLA/PDLA. PDLA is used as a nucleation agent that increases the crystallinity, melting temperature and enhances other physical properties.

The integration of PDLA causes an issue for all PLA recycling process that hydrolyze the polymer (thermally, chemically or enzymatically) into lactic acid or lactide monomers. A need arises to separate the isomers in order to produce pure L-lactate or L-L-lactide stereoisomers.

In the food waste to PLA process, PLA waste can be integrated with the food waste by chemical, thermal or enzymatic hydrolysis into lactic acid monomers, and added to the same processing tank that contains the D-lactate oxidase. The enzyme then eliminates both the D-lactic acid naturally found in the waste (originating in natural fermentation decay processes in garbage bins, storage and transportation), and the D-lactate recycled from PLA waste. This method can significantly increase the titer and output of lactic acid from a facility, and improve facility techno-economics without massive investments in new equipment and operational expenditure (utilities, reagents).

As used herein, the term “about”, when referring to a measurable value, is meant to encompass variations of +/−10%, preferably +/−5%, more preferably, +/−1%, and still more preferably +/−0.1% from the specified value.

The terms “comprises”, “comprising”, “includes”, “including”, “having” and their conjugates mean “including but not limited to”. The terms “comprises” and “comprising” are limited in some embodiments to “consists” and “consisting”, respectively. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Production of a Recombinant D-lactate Oxidase From Gluconobacter oxydans

A pET30 plasmid encoding D-lactate oxidase (DOX) from Gluconobacter oxydans strain 621H was constructed. The nucleotide sequence encoding the DOX (SEQ ID NO: 2) was modified to favor expression in E. coli. The DOX coding sequence modified for expression in E. coli is set forth as SEQ ID NO: 9. The enzyme was expressed as a non-secreted protein, with a His-tag.

An overnight culture of Escherichia coli BL21 (DE3)/pET30-dox cells was inoculated in 1L TB medium (Tryptone 12 g/l, yeast extract 24 g/l, glycerol 4 ml/l, KH₂PO₄-2.3 g/l, K₂HPO₄-16.43 g/l) containing kanamycin (50 ug/ml) to an O.D600 of 0.1. The vessel was then incubated at 37° C. with agitation until O.D600 reached 1.2. Next, the vessel was transferred to 15° C. and IPTG was added at a final concentration of 0.5 mM to induce expression of the enzyme. Following 16 hours incubation at 15° C., bacterial cells were collected by centrifugation, suspended in lysis buffer (50 mM TRIS-HCl, 150 mM NaCl, 10% glycerol, pH=8) and disrupted by sonication.

Bacterial lysate was centrifuged and the resulting supernatant was loaded onto a Ni column and washed with wash buffer (50 mM Tris-HCl, 10% glycerol, pH=8). The protein was eluted with wash buffer containing 300 mM Imidazole. The eluted protein was dialyzed against lysis buffer and stored in −80° C. until activity was evaluated.

### Example 2

### Degradation of Pure D-lactate and D-lactate Present in Organic Waste by the Recombinant D-lactate Oxidase

Experiments were carried out to characterize the activity of the enzyme. It was previously reported that the optimum pH for the enzyme is between 7-9 (best activity was seen at pH=8) and that the optimum temperature is 55° C. (Sheng et al. 2016, supra, Supporting Information, FIG. S3). Thus, the initial experiments carried out with the enzyme were conducted at 55° C., pH=8.

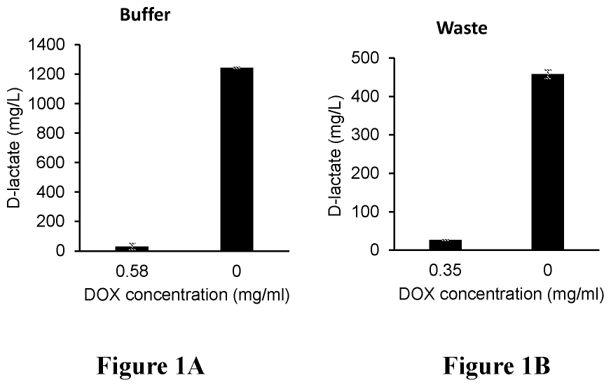

First, the activity was tested with pure D-lactate dissolved in a buffer. A solution of 1200 mg/L D-lactate dissolved in buffer solution containing 50 mM Tris-HCl, 150 mM NaCl, 10% Glycerol, pH 8.0 was incubated with the enzyme (0.58 mg/ml) for 24 hours. A buffer solution without the enzyme was used as a control. As shown in FIG. 1A, 0.58 mg/ml of the enzyme consumed substantially all the available D-lactate after 24 hours of incubation.

Next, the activity of the enzyme was tested on organic waste containing both D- and L-lactic acid. The organic waste was a mixed food waste containing bakery factory rejects, fruit, vegetables and butchery waste (dark meat), and waste dairy products. The waste was ground and diluted 1:1 with water and incubated with the enzyme (0.35 mg/ml) for 24 hours. Waste without the enzyme was used as a control. The diluted waste contained a total of 1250 mg/L lactate, of which 450 mg/L were D-lactate. The total lactate was measured using the Reflectoquant® lactic acid test system (Merck). The D-lactate content was measured using a specific D-lactate colorimetric kit (Sigma).

As shown in FIG. 1B, 0.35 mg/ml of the enzyme consumed approximately 95% of the D-lactate in the waste: the initial concentration of D-lactate in the waste was 450 mg/L, and after 24 hours of incubation with the enzyme the concentration was only 26 mg/L. Even though the organic waste is a complex viscous substrate the exact composition of which is unknown, with possible inhibitors and other factors that could negatively affect the enzyme, as well as unknown amounts of the co-factor that is needed for its activity, which may not be sufficient, the enzyme was able to effectively eliminate D-lactate.

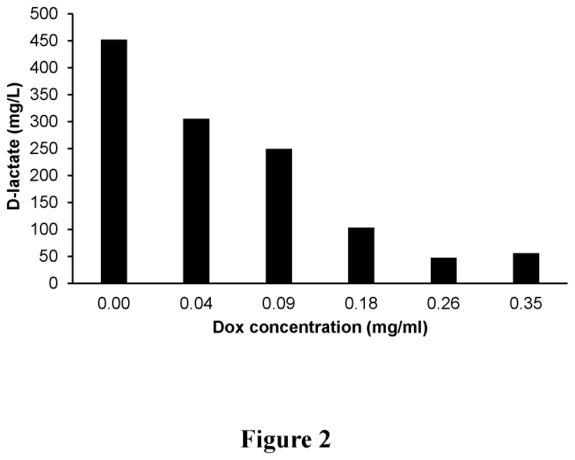

The activity of the enzyme on organic waste was further tested using different concentrations of the enzyme. Organic waste containing 450 mg/L D-lactate (after dilution 1:1 with water) was incubated for 24 hours with different concentrations of the enzyme, as follows: 0.35, 0.26, 0.18, 0.09 or 0.04 mg/ml. Waste without the enzyme was used as a control. As shown in FIG. 2, 0.35, 0.26, 0.18, 0.09 or 0.04 mg/ml of the enzyme consumed approximately 87%, 89%, 77%, 44% or 32%, respectively, of the available D-lactate. Under the conditions tested in this experiment, a minimal concentration of 0.26 mg/ml of DOX was needed in order to achieve a significant decrease in D-lactate in the waste after 24 h.

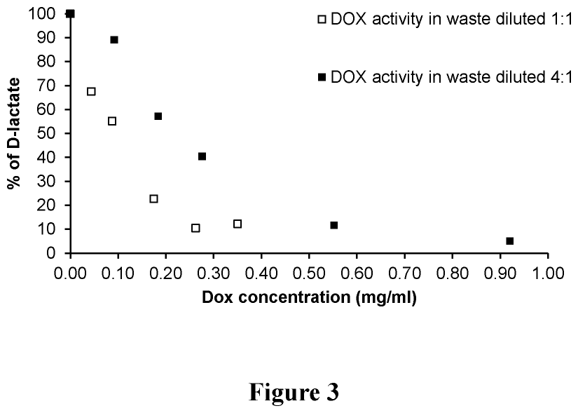

In the above experiments the organic waste was diluted 1:1 with water before the addition of the enzyme (400 μl of the waste were diluted with 400 μl of water). In the following experiment the activity of the enzyme on a waste diluted 1:1 with water versus 4:1 was examined. The 4:1 dilution was achieved by mixing 400 μl of the waste with 100 μl of water. The amount of D-lactate in the waste after 24 hours incubation with increasing concentrations of the enzyme was examined. The results are summarized in FIG. 3. As shown in the figure, 24 hours of incubation at 55° C. were sufficient for a concentration of 0.27 mg/ml DOX to consume approximately 90% of the D-lactate in the 1:1 diluted waste, while 0.55 mg/ml were needed for the waste that was diluted 4:1. These results may indicate that the enzyme needs a more diluted environment for efficient consumption of D-lactate. It may also indicate the need for a more efficient mixing of the waste (as the experiment was carried out in test tubes the shaking may not be optimal).

### Example 3

### Activity of the D-lactate Oxidase in the Presence of a Glucoamylase

The following experiment examined how the presence of a glucoamylase (GA) affects the activity of the DOX. The glucoamylase that was used is a commercially available glucoamylase from Aspergillus niger.

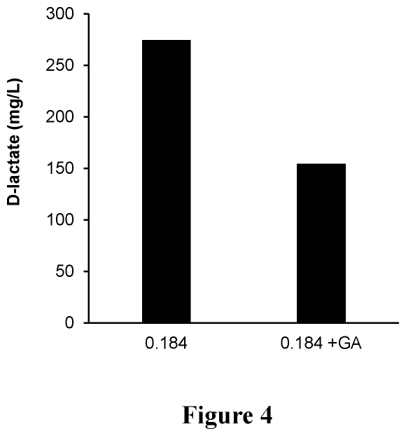

An organic waste diluted 4:1 with water was incubated for 24 hours at 55° C. pH=8 with 0.18 mg/ml DOX, with or without 100 units/ml GA. Although pH=8 is not ideal for the GA, at this point it was not known whether the DOX would be active at an acidic pH. The initial concentration of D-lactate in the waste was 959 mg/L. When DOX was added with the GA, 154 mg/L D-lactate were left in the tube, while without GA, 274 mg/L were left (FIG. 4). These results indicate that a combination of DOX with a polysaccharide-degrading enzyme such as a glucoamylase is advantageous and results in improved activity of the DOX. When the DOX is combined with a polysaccharide-degrading enzyme such as a glucoamylase, the process requires less dilution of the substrate (the organic waste) and can be carried out using lower amounts of the DOX. Without wishing to be bound by any particular theory or a mechanism of action, it is contemplated that the improved activity of the DOX in the presence of a GA stems from the reduced viscosity of the waste upon degradation of starch present in the waste by the GA into soluble sugars.

### Example 4

### Activity of the D-lactate Oxidase at Different pH and Temperatures

As noted above, it was previously reported that the enzyme works best at pH=8 and 55° C. (Sheng et al. 2016, Supporting Information, FIG. S3). With respect to pH, it was shown that the enzyme's activity and stability is significantly reduced at pH lower than 7. However, organic waste is typically acidic due to natural decay processes in garbage bins, storage and transportation (pH ranging between 4 to 5.5). In addition, for lactic acid production from the organic waste, the organic waste is saccharified by polysaccharide-degrading enzymes such as amylases and cellulases, which are typically active at acidic pH values. With respect to temperature, some lactic acid production processes are carried out by mesophilic bacteria. It was thus decided to check the enzyme's activity at varying pH and temperatures, even at pH and temperatures in which the enzyme was reported to lose activity/stability.

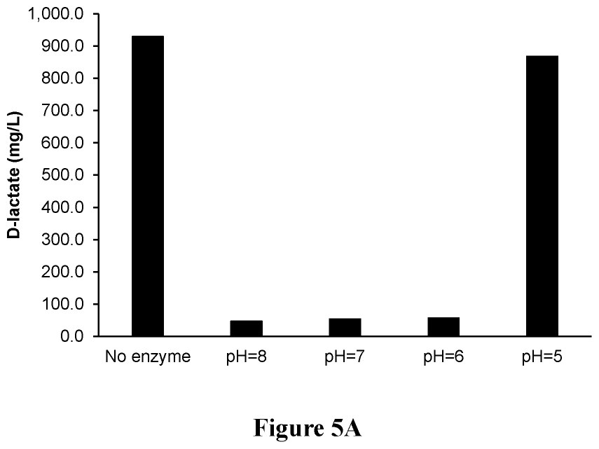

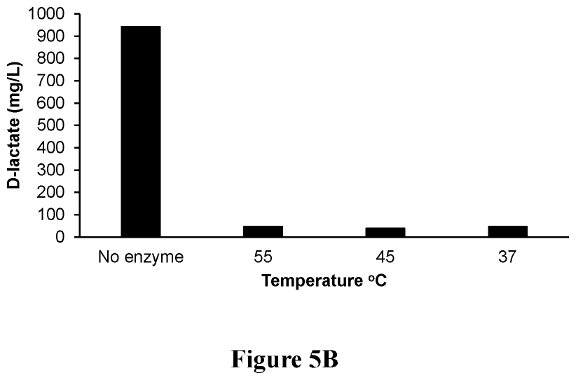

First, activity at varying pH were tested. To this end, the waste was adjusted to pH=5, 6, 7 or 8 and 0.27 mg/ml enzyme was added and incubated at 55° C. for 24 hours. The results are summarized in FIG. 5A. As shown in the figure, the enzyme surprisingly showed effective elimination of D-lactate even at pH=7 and pH=6. In effect, the activity of the enzyme was substantially the same as the activity at pH=8, and the change towards a more acidic pH did not impair its activity. Only at pH=5 the activity of the enzyme was impaired and substantially no D-lactate was eliminated from the medium.

Next, a similar experiment was carried out in which the enzyme was tested at different temperatures. The enzyme (0.27 mg/ml) was added to the waste and incubated at three different temperatures for 24 hours. The results are summarized in FIG. 5B. As shown in the figure, the amount of D-lactate remaining at the end of the experiment was substantially the same for all the tested temperatures.

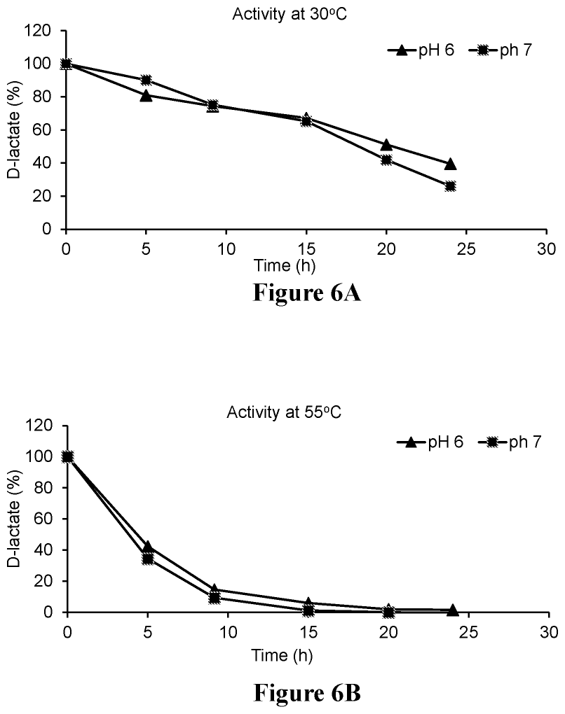

In further experiments the enzyme's activity was examined in the supernatant of organic waste at varying temperatures and pH. In the first experiment, the waste was centrifuged (9000 g 10 min) prior to adjustment of the pH to pH 7.0 or pH 6.0 and incubated with the enzyme (0.27 mg/ml) at 30° C. As presented in FIG. 6A the trend of the activity of the enzyme at pH 6.0 and 7.0 were very similar at 30° C. The D-lactate decreased by 60% or 75% respectively (from ˜1800 mg/L to 700 mg/L or 470 mg/L respectively), indicating a slight advantage to pH 7.0 at this temperature. In an additional experiment, the waste was centrifuged (9000 g 10 min) prior to adjustment of the pH to pH 7.0 or pH 6.0 and incubated with the enzyme (0.27 mg/ml) at 55° C. As presented in FIG. 6B the activity of the enzyme was better at 55° C. compared to 33° C., and the trend of the activity of the enzyme at pH 6.0 and 7.0 were very similar. All the D-lactate in the waste (˜1800 mg/L) was consumed at both pH values, indicating that at this temperature both pH values can be used.

The assays in Sheng et al. supra were carried out in a buffer solution (50 mM Tris-HCl, 150 mM NaCl, 10% Glycerol, pH 8.0). The pH and temperature results described herein show improved activity of the enzyme in organic waste compared to its activity in a buffer solution, characterized by a broader range of conditions in which the enzyme is active and effectively eliminates D-lactate. These results indicate that the enzyme is useful for industrial processing and fermentation of a variety of organic wastes of different pH, and also that the enzyme may potentially be used at different time points in a process for producing lactic acid from organic waste, and in combination with other steps such as saccharification of the waste.

### Example 5

### Degradation of D-lactate Present in Organic Waste Before and After Lactic Acid Fermentation

The following experiment tested the D-lactate oxidase on organic waste obtained from a 15,000 liter L-lactic acid production line in which organic waste is used as the substrate for fermentation. The organic waste was a mixed food waste combining bakery rejects and market returns of fruit, vegetables and milk. The waste was pretreated by mixing, shredding, grinding and steam injection.

The activity of the D-lactate oxidase was tested on samples that were taken from the organic waste at two time points—before and after fermentation. Before fermentation the D-lactate concentration was approximately 2200 mg/L. After fermentation the concentration of D-lactate remained substantially the same (slightly reduced due to dilution of the waste following inoculation of the bacteria and addition of reagents such as pH control reagents during fermentation). During the process the L-lactate concentration was increased to approximately 80,000 mg/L. It was of interest to examine the activity of the D-lactate oxidase in the presence of such a significant excess of L-lactic acid compared to D-lactic acid.

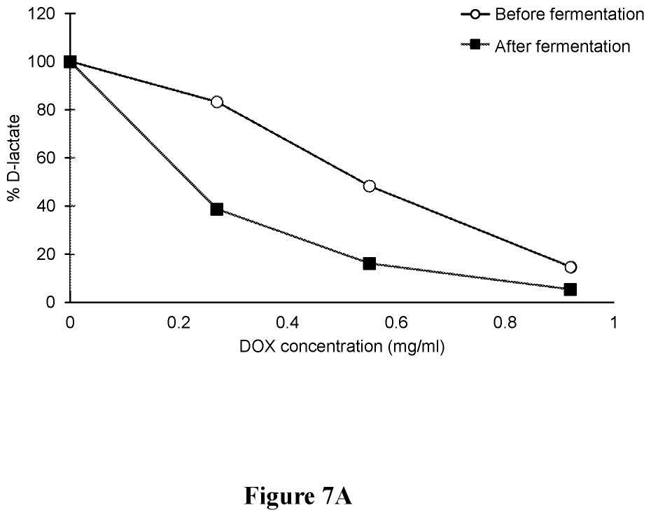

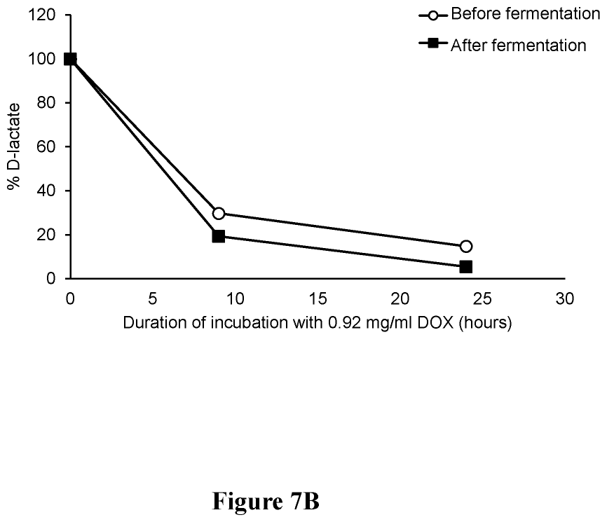

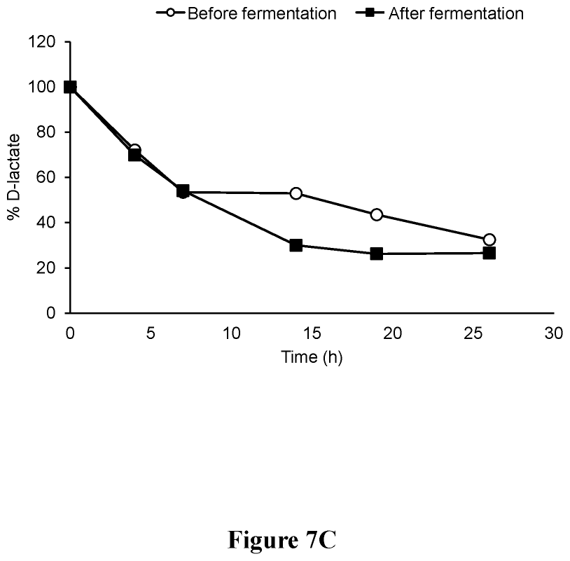

The two samples were incubated with different concentrations of the D-lactate oxidase for 24 hours at 55° C. A concentration of 0.92 mg/ml was also tested in a 9-hour incubation with the organic waste. The sample that was taken before fermentation was incubated with the D-lactate oxidase without adjusting the pH, which was 5.5. The sample that was taken after fermentation had a pH=6.4. Samples with no enzyme were used as a control. Following incubation with the enzyme the D-lactate concentration was measured. The results of the 24-hour incubation with different concentrations of the enzyme are summarized in Table 1 and FIG. 7A. The figure presents the results as percentage of the D-lactate concentration in the control reaction (“0” enzyme).

TABLE 1
D-lactate concentration following a 24
h incubation with the D-lactate oxidase

| | Waste sample | |
|---|---|---|
| Enzyme con. | “Before fermentation” | “After fermentation” |
| 0 | 2168 mg/L | 1833 mg/L |
| 0.27 mg/ml | 1804 mg/L | 710 mg/L |
| 0.55 mg/ml | 1048 mg/L | 297 mg/L |
| 0.92 mg/ml | 320 mg/L | 99 mg/L |

In the sample that was taken before fermentation a concentration of 0.92 mg/ml of the enzyme was able to efficiently consume the D-lactate present in the waste after 24 hours of incubation—the D-lactate concentration was reduced by 85%. Interestingly, in the sample that was taken after fermentation a lower concentration of the enzyme, 0.55 mg/ml, was sufficient to effectively consume the D-lactate present in the waste and reduce its concentration by about 85% after 24 hours. The improved activity of the enzyme on the sample taken after fermentation could be because the waste after fermentation is less viscous compared to its viscosity before fermentation and treatment with a glucoamylase. In addition, the sample taken after fermentation had a pH=6.4, which could be more suitable for the D-lactate oxidase compared to pH=5.5 of the sample taken before fermentation. The concentration of the D-lactate after incubation with the enzyme was 297 mg/ml while the concentration of the L-lactate after fermentation was 80,000 mg/ml. Thus, the D-lactate oxidase was able to reduce the amount of D-lactate such that it is less than 0.5% of the total lactate at the end of fermentation. Importantly, the enzyme was able to do so even in the presence of a significant excess of L-lactate compared to D-lactate.

The change in D-lactate in the waste using 0.92 mg/ml of the D-lactate oxidase was measured following 9 hours of incubation in addition to the 24-hour measurement discussed above. FIG. 7B shows the change in D-lactate over time following incubation with 0.92 mg/ml of the D-lactate oxidase. The figure presents the results as percentage of the D-lactate concentration at time=0. The D-lactate oxidase at a concentration of 0.92 mg/ml incubated with the sample taken after fermentation was able to consume about 80% of the D-lactate already after 9 hours.

In a further experiment, the activity of the D-lactate oxidase was tested on a different source of mixed food organic waste that was used as a substrate for fermentation, containing bakery rejects, fruit, vegetables, butchery waste (dark meat) and waste dairy products. The waste was pretreated by mixing, grinding and sterilization. The activity of the D-lactate oxidase was tested on samples that were taken from the organic waste at two time points—before and after lactic acid fermentation. In this experiment, the samples were centrifuged (9000 g 10 min) prior to incubation with the D-lactate oxidase. Following centrifugation, 0.27 mg/ml of the enzyme were added to the supernatant and incubated at 55° C. for 26 hours. The concentration of D-lactate was measured at 0, 4, 7, 14, 19 and 26 hours of incubation.

The D-lactate concentration in the sample taken before fermentation was approximately 800 mg/L. After fermentation the concentration of D-lactate remained substantially the same. During the process the L-lactate concentration was increased to approximately 87,000 mg/L. Again, it was of interest to examine the activity of the D-lactate oxidase in the presence of such a significant excess of L-lactic acid compared to D-lactic acid.

Following incubation with the enzyme the D-lactate concentration was measured. The results are summarized in Table 2 and FIG. 7C. The figure presents the results as percentage of the D-lactate concentration at time=0.

TABLE 2
D-lactate concentration following incubation of
waste supernatant with the D-lactate oxidase

| | Waste sample | |
|---|---|---|
| Enzyme con. | “Before fermentation” | “After fermentation” |
| 0 | 811 mg/L | 846 mg/L |
| 4 | 584 mg/L | 591 mg/L |
| 7 | 433 mg/L | 457 mg/L |
| 14 | 429 mg/L | 253 mg/L |
| 19 | 353 mg/L | 221 mg/L |
| 26 | 263 mg/L | 224 mg/L |

In the sample that was taken before fermentation a concentration of 0.27 mg/ml of the enzyme was able to consume approximately 70% of the D-lactate present in the waste after 26 hours of incubation. Interestingly, in the sample that was taken after fermentation the same concentration was able to consume the same amount of D-lactate after only 14 hours. Since the waste was centrifuged prior to the incubation with the D-lactate oxidase, it seems that in addition to reduced viscosity, other factors bring to improved activity after the fermentation is completed.

The concentration of the D-lactate after incubation with the enzyme was 224 mg/ml while the concentration of the L-lactate after fermentation was 87,000 mg/ml. Thus, the D-lactate oxidase was able to reduce the amount of D-lactate such that it is less than 0.5% of the total lactate at the end of fermentation. Again, the enzyme was able to do so even in the presence of a significant excess of L-lactate compared to D-lactate.

### Example 6

### Improved Protocol for the Production of the Recombinant D-lactate Oxidase

An overnight culture of E. coli BL21 (DE3)/pET30-dox cells described in Example 1 was inoculated in 5 mL LB medium containing kanamycin (50 ug/ml) and grown overnight. Next, 1 mL of the culture was inoculated to 50 mL of TB medium (Tryptone 12 g/l, yeast extract 24 g/l, glycerol 4 ml/l, KH₂PO₄-2.3 g/1, K2HPO4 -16.43 g/l) containing kanamycin and was incubated at 37° C. with agitation until O.D600 reached 1.2. The culture was then transferred to 15° C. and IPTG was added at a final concentration of 0.5 mM to induce expression of the enzyme. Following 20 hours incubation at 15° C., bacterial cells were collected by centrifugation, suspended in lysis buffer (50 mM TRIS-HCl, 150 mM NaCl, 10% glycerol, pH=8) and disrupted by sonication.

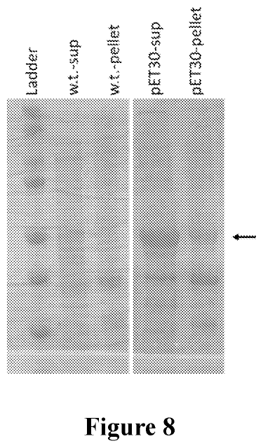

Bacterial lysate was centrifuged at 3000 rpm for 5 min and the supernatant was centrifuged again at 9000 rpm for 10 min. The resulting pellet and the supernatant were mixed with sample buffer, boiled, and visualized on SDS-PAGE. The soluble active form of the protein is found in the supernatant, while non-active protein aggregates are found in the pellet. As shown in FIG. 8, the vast majority of the protein was found in the supernatant, indicating successful expression of the protein in an active soluble form. The amount of protein in the soluble (supernatant) fraction was significantly higher than that achieved using the protocol described in Example 1.

The crude supernatant is tested for degradation of D-lactate as described above.

### EXAMPLE 7

### Production of the Recombinant D-lactate Oxidase From Gluconobacter oxydans Using Constitutive Promoters

pET30 plasmids expressing the D-lactate oxidase (DOX) from Gluconobacter oxydans strain 621H under constitutive promoters were constructed. The nucleotide sequence encoding the DOX (SEQ ID NO: 2) was modified to favor expression in E. coli. The modified sequence is set forth as SEQ ID NO: 9. The enzyme was expressed as a non-secreted protein, with a His-tag.

Three types of plasmids were constructed, each containing one of the following synthetic promoters cloned upstream to the DOX coding sequence, replacing the inducible T7 promoter:

>SEQ ID NO: 3:
AAGCTGTTGTGACCGCTTGCTCTAGCCAGCTATCGAGTTGTGAACCGA
TCCATCTAGCAATTGGTCTCGATCTAGCGATAGGCTTCGATCTAGCTA
TGTAGAAACGCCGTGTGCTCGATCGCTTGATAAGGTCCACGTAGCTGC
TATAATTGCTTCAACAGAACATATTGACTATCCGGTATTACCCGGC
>SEQ ID NO: 4:
CTTGATAAGGTCCACGTAGCTGCTATAGTTGCTTCAACAGAACATATT
GACTATCCGGTATTACCCGGC
>SEQ ID NO: 5:
CCTGATAAGGTCCACAGTAGCTGCTATAATTGCTTCAACAGAACATAT
TGACTATCCGGTATTACCCGGC

Nucleic acid constructs containing the nucleic acid sequence encoding the DOX modified for expression in E. coli along with each one of the above promoters is set forth as:
SEQ ID NO: 6—a nucleic acid construct comprising the promoter sequence set forth as SEQ ID NO: 3. The promoter sequence corresponds to positions 1-190 of SEQ ID NO: 6. The DOX coding sequence corresponds to positions 256-1,692 of SEQ ID NO: 6. The nucleic acid construct further contains a nucleic acid sequence encoding an N-terminal His-tag and a Met residue upstream to the His-tag at positions 235-255.
SEQ ID NO: 7—a nucleic acid construct comprising the promoter sequence set forth as SEQ ID NO: 4. The promoter sequence corresponds to positions 1-69 of SEQ ID NO: 7. The DOX coding sequence corresponds to positions 135-1,571 of SEQ ID NO: 7. The nucleic acid construct further contains a nucleic acid sequence encoding an N-terminal His-tag and a Met residue upstream to the His-tag at positions 114-134.
SEQ ID NO: 8—a nucleic acid construct comprising the promoter sequence set forth as SEQ ID NO: 5. The promoter sequence corresponds to positions 1-70 of SEQ ID NO: 8. The DOX coding sequence corresponds to positions 136-1,572 of SEQ ID NO: 8. The nucleic acid construct further contains a nucleic acid sequence encoding an N-terminal His-tag and a Met residue upstream to the His-tag at positions 115-135.

Each of the above nucleic acid constructs further contains a ribosome binding site between the promoter sequence and the DOX coding sequence.

For each type of plasmid, an overnight culture of E. coli BL21 (DE3) transformed with the plasmid was inoculated in 1L TB medium (Tryptone 12 g/l, yeast extract 24 g/l, glycerol 4 ml/l, KH₂PO₄-2.3 g/l, K₂HPO₄-16.43 g/l) containing kanamycin (50 ug/ml) and agitated at 37° C. for 16 hours. Bacterial cells were collected by centrifugation, suspended in lysis buffer (50 mM TRIS-HCl, 150 mM NaCl, 10% glycerol, pH=8) and disrupted by sonication.

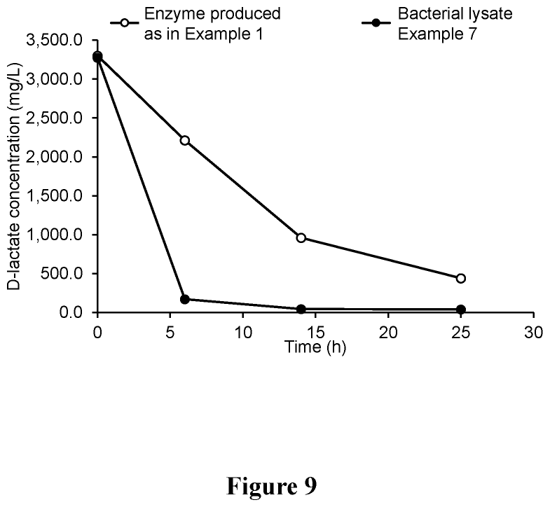

Bacterial lysates of E. coli expressing the D-lactate oxidase under a constitutive promoter were tested for degradation of D-lactate. The D-lactate oxidase produced in Example 1 (0.27 mg/ml) was used as a control. A lysate was added as 20% (v/v) of the total reaction volume: 20 ul of a lysate containing the enzyme were added to 80 ul of a buffer containing about 3,200 mg/L D-lactate, pH=8, and incubated at 55° C. The activity was measured for a total of 25 hours. The results are shown in FIG. 9. After 25 hours the enzyme from Example 1 was able to decrease the D-lactate to 400 mg/L, while the bacterial lysate was able to eliminate almost all the D-lactate after only 6 hours (170 mg/L).

### Example 8

### Simultaneous Fermentation, Saccharification and D-lactate Elimination

### A. Simultaneous Process: Glucoamylase+DOX+Bacillus coagulans

Waste stream is ground, added to a fermenter and sterilized. A glucoamylase (0.1-1 gr/L), DOX (0.25-0.65 mg/ml) and B. coagulans (10\hat{ }6-10\hat{ }8 live bacterial cells) are added at a temperature of 50-55° C. and pH=5.5-6.5. A sample is taken from the waste before its addition to the fermenter for assessment of glucose potential and measurement of initial D-lactate concentration. Samples are also taken during the fermentation process, for example every 1-10 hours or every 1-5 hours, to monitor glucose and total lactate concentrations. The fermentation continues until the glucose concentration reaches zero and there is no more increase in total lactate concentration. At this point, which takes between 20-48 hours to reach, the D-lactate concentration decreases to levels which are less than 0.5% of the final total lactate.

### B. Simultaneous Process: Glucoamylase+Bacillus coagulans+DOX Added in “Spikes”

Waste stream is ground, added to a fermenter and sterilized. Glucoamylase (0.1-1 gr/L), DOX (0.1-0.3 mg/ml) and B. coagulans (10\hat{ }6-10\hat{ }8 live bacterial cells) are added at a temperature of 50-55° C. and pH=5.5-6.5. Five (5) hours later a second dose of DOX is added (0.1-0.3 mg/ml). 5 hours later a third dose of DOX is added (0.1-0.3 mg/ml). 5 hours later a fourth dose of DOX is added (0.1-0.3 mg/ml). A sample is taken from the waste before its addition to the fermenter for assessment of glucose potential and measurement of initial D-lactate concentration. Samples are also taken during the fermentation process, for example every 1-10 hours or every 1-5 hours, to monitor glucose and lactate concentrations. The fermentation continues until the glucose concentration reaches zero and there is no more increase in lactate concentration. At this point, which takes between 20-48 hours to reach, the D-lactate concentration decreases to levels which are less than 0.5% of the final total lactate.

### C. Simultaneous Saccharification and Fermentation, With DOX Added at the End of Fermentation

Waste stream is ground, added to a fermenter and sterilized. Glucoamylase (0.1-1 gr/L) and B. coagulans (10\hat{ }6-10\hat{ }8 live bacterial cells) are added at a temperature of 50-55° C. and pH=5.5-6.5. A sample is taken from the waste before its addition to the fermenter for assessment of glucose potential and measurement of initial D-lactate concentration. Samples are also taken during the fermentation process, for example every 1-10 hours or every 1-5 hours, to monitor glucose and total lactate concentrations. The fermentation continues until the glucose concentration reaches zero and there is no more increase in total lactate concentration. At this point, which takes between 20-48 hours to reach, the fermentation broth is centrifuged (9000 g, 10 min) and the solid phase is discarded. DOX enzyme is added to the broth supernatant (0.25-0.65 mg/ml) and the supernatant is incubated for 10-24 hours at 50-55° C. and pH=6-7. The incubation continues until the D-lactate concentration decreases to levels which are less than 0.5% of the total lactate.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-46. (canceled)

47. A method for processing organic waste, the method comprising:
(i) providing an organic waste; and
(ii) digesting the organic waste with a D-lactate oxidase, to eliminate D-lactic acid present in the organic waste, wherein the organic waste is selected from the group consisting of food waste, municipal waste, agricultural waste, plant material and combinations thereof.

48. The method of claim 47, wherein the organic waste is food waste.

49. The method of claim 47, wherein the D-lactate oxidase is from Gluconobacter oxydans.

50. The method of claim 49, wherein the digesting of the D-lactate oxidase with the organic waste is carried out at a temperature in the range of 25-60° C.

51. The method of claim 49, wherein the digesting of the D-lactate oxidase with the organic waste is carried out at a pH in the range of 5.5-7.

52. The method of claim 47, wherein the digesting of the D-lactate oxidase with the organic waste is carried out for a time period ranging from 6 to 48 hours.

53. The method of claim 47, further comprising contacting the organic waste with one or more saccharide-degrading enzyme, to degrade saccharides in the organic waste to release reducing sugars, wherein the contacting with the one or more saccharide-degrading enzyme and the digesting with the D-lactate oxidase are carried out simultaneously or sequentially in any order.

54. The method of claim 53, wherein the one or more saccharide-degrading enzyme is a polysaccharide-degrading enzyme selected from the group consisting of an amylase, a cellulase and a hemicellulose.

55. The method of claim 53, wherein the one or more saccharide-degrading enzyme comprises a glucoamylase.

56. A method for producing L-lactic acid from organic waste, the method comprising:
(i) processing the organic waste using a D-lactate oxidase according to claim 47, to eliminate D-lactic acid originating from the organic waste; and
(ii) fermenting the organic waste with a lactic acid-producing microorganism that produces only L-lactic acid,
wherein eliminating D-lactic acid originating from the organic waste using a D-lactate oxidase is carried out prior to, after or simultaneously with fermenting the organic waste with a lactic acid-producing microorganism that produces only L-lactic acid.

57. The method of claim 56, wherein the organic waste is food waste.

58. The method of claim 56, wherein the D-lactate oxidase is from Gluconobacter oxydans.

59. A method for producing L-lactic acid from organic waste, the method comprising:
(i) providing an organic waste;
(ii) processing the organic waste to eliminate D-lactic acid present in the waste and degrade saccharides in the waste to release soluble reducing sugars, by contacting the organic waste with a D-lactate oxidase and one or more saccharide-degrading enzyme, wherein the contacting with the one or more saccharide-degrading enzyme and the contacting with the D-lactate oxidase are carried out simultaneously or sequentially in any order;
(iii) fermenting the processed organic waste with a lactic acid-producing microorganism that produces only L-lactic acid, to obtain L-lactic acid; and
(iv) recovering the L-lactic acid from the fermentation broth.

60. The method of claim 59, wherein the organic waste is food waste.

61. The method of claim 59, wherein the D-lactate oxidase is from Gluconobacter oxydans.

62. The method of claim 59, wherein the one or more saccharide-degrading enzyme comprises a glucoamylase.

63. The method of claim 59, wherein step (ii) and step (iii) are carried out simultaneously.

64. The method of claim 59, wherein step (ii) is carried out prior to step (iii).

65. A nucleic acid construct for expressing a D-lactate oxidase, comprising a nucleic acid sequence encoding a D-lactate oxidase as set forth in SEQ ID NO: 9, operably linked to at least one regulatory sequence comprising a promoter selected from the group consisting of: SEQ ID NO: 3, SEQ ID NO: 4 and SEQ ID NO: 5.

66. The nucleic acid construct of claim 65, selected from the group consisting of SEQ ID NO: 6, SEQ ID NO: 7 and SEQ ID NO: 8.


SEQUENCE LISTING (9 sequences)

SEQ ID NO: 1 - protein, 477 aa, Gluconobacter oxydans
MPEPVMTASSASAPDRLQAVLKALQPVMGERISTAPSVREEHSHGEAMNASNLPEAVVFAESTQDVATVLRHCHEWRVPVVAFGAGTSVEGHVVPPEQAISLDLSRMTGIVDLNAEDLDCRVQAGITRQTLNVEIRDTGLFFPVDPGGEATIGGMCATRASGTAAVRYGTMKENVLGLTVVLATGEIIRTGGRVRKSSTGYDLTSLFVGSEGTLGIITEVQLRLHGRPDSVSAAICQFESLHDAIQTAMEIIQCGIPITRVELMDSVQMAASIQYSGLNEYQPLTTLFFEFTGSPAAVREQVETTEAIASGNNGLGFAWAESPEDRTRLWKARHDAYWAAKAIVPDARVISTDCIVPISRLGELIEGVHRDIEASGLRAPLLGHVGDGNFHTLIITDDTPEGHQQALDLDRKIVARALSLNGSCSGEHGVGMGKLEFLETEHGPGSLSVMRALKNTMDPHHILNPGKLLPPGAVYTG

SEQ ID NO: 2 - DNA, 1434 bp, Artificial Sequence
  Polynucleotide
atgccggaaccagtcatgaccgcctcttccgcctccgctccggaccgccttcaggccgttctcaaagccctccagcccgtcatgggtgagcggatcagcacggcaccctccgttcgcgaagagcacagccacggcgaggccatgaatgcctccaacctgcccgaggcggtggtgtttgctgaaagtactcaggatgtcgcaaccgtcctgcggcactgccatgaatggcgcgttccggtcgtggcgttcggcgctggcacgtccgtcgaaggtcatgtcgtgccgcccgaacaggccatcagcctcgatctgtcacgcatgacggggatcgtggacctgaacgccgaggatctggattgccgggtccaagccggcatcacgcgccagacgctgaatgttgaaatccgcgatacgggcctgttctttccggtcgatccgggtggggaagctacgatcggcggtatgtgcgccacccgcgcctcgggcacggccgccgtacgctacggcacgatgaaagaaaatgtgctgggcctgacggttgttctcgcgaccggcgaaatcatccgcacaggtggccgcgtccgcaaatcgtccaccggctatgacctgacatcgctgttcgtcggctcggaaggtacgctcgggatcatcaccgaagtccagctccgtctgcatgggcgtccagacagtgtttcggccgcgatctgccaattcgaaagcctgcatgacgccatccagactgccatggaaatcatccagtgcggcatccccatcacccgcgtggaactgatggacagcgtgcagatggcagcttccatccagtattccggcctgaacgaatatcagccgctgaccacgctgtttttcgagttcacaggctcgcccgcagcggtacgcgagcaggtcgagacgaccgaagccattgcgtccggcaataacgggcttggctttgcctgggccgaaagtcccgaagaccgcacccgcctctggaaagcgcggcatgacgcctactgggcggccaaggccatcgttccggatgcgcgcgtcatttccacagactgcatcgtcccgatttcccgtctgggcgaactgatcgagggcgtgcatcgcgatatcgaggcctccggcctgcgcgcgccccttctgggccatgtgggggacggcaatttccatacgctcatcatcacggacgacacccccgaagggcatcagcaggccctcgatctggaccggaagatcgtagcccgcgccctttcgctgaacgggtcgtgcagcggggaacatggtgtcggcatgggcaagctggagtttctggaaaccgagcatgggcctggaagcctcagcgtgatgcgcgccctgaagaacacgatggatccgcaccatatcctcaatcccggcaagctccttccgcccggtgctgtttacacgggctga

SEQ ID NO: 3 - DNA, 190 bp, Artificial Sequence
  Promoter
aagctgttgtgaccgcttgctctagccagctatcgagttgtgaaccgatccatctagcaattggtctcgatctagcgataggcttcgatctagctatgtagaaacgccgtgtgctcgatcgcttgataaggtccacgtagctgctataattgcttcaacagaacatattgactatccggtattacccggc

SEQ ID NO: 4 - DNA, 69 bp, Artificial Sequence
  Promoter
cttgataaggtccacgtagctgctatagttgcttcaacagaacatattgactatccggtattacccggc

SEQ ID NO: 5 - DNA, 70 bp, Artificial Sequence
  Promoter
cctgataaggtccacagtagctgctataattgcttcaacagaacatattgactatccggtattacccggc

SEQ ID NO: 6 - DNA, 1692 bp, Artificial Sequence
  Polynucleotide
aagctgttgtgaccgcttgctctagccagctatcgagttgtgaaccgatccatctagcaattggtctcgatctagcgataggcttcgatctagctatgtagaaacgccgtgtgctcgatcgcttgataaggtccacgtagctgctataattgcttcaacagaacatattgactatccggtattacccggccctctagaaataattttgtttaactttaagaaggagatatacatatgcaccaccaccaccaccacatgcccgaaccagtaatgacagcttcaagcgcgagcgcgccggatcgtctgcaagcggttctgaaggcgctgcagccggtgatgggtgaacgtatcagcaccgcgccgagcgttcgtgaggaacacagccacggcgaggcgatgaacgcgagcaacctgccggaagcggtggtttttgcggagagcacccaggatgtggcgaccgttctgcgtcactgccacgaatggcgtgtgccggtggttgcgtttggtgcgggcaccagcgttgaaggtcacgtggttccgccggagcaagcgatcagcctggacctgagccgtatgaccggcattgtggatctgaacgcggaggacctggattgccgtgttcaggcgggtatcacccgtcaaaccctgaacgtggaaattcgtgacaccggcctgttctttccggttgatccgggtggcgaggcgaccatcggtggcatgtgcgcgacccgtgcgagcggtaccgcggcggtgcgttacggcaccatgaaggaaaacgttctgggtctgaccgtggttctggcgaccggcgagatcattcgtaccggtggccgcgtgcgtaaaagcagcaccggttatgacctgaccagcctgttcgttggcagcgaaggtaccctgggcatcattaccgaggtgcaactgcgtctgcatggccgtccggacagcgttagcgcggcgatctgccaatttgaaagcctgcacgatgcgattcagaccgcgatggagatcattcaatgcggtatcccgattacccgtgtggaactgatggatagcgttcagatggcggcgagcatccaatacagcggtctgaacgaatatcagccgctgaccaccctgttctttgagtttaccggcagcccggcggcggtgcgtgagcaagttgaaaccaccgaggcgattgcgagcggtaacaacggtctgggctttgcgtgggcggaaagcccggaggaccgtacccgtctgtggaaggcgcgtcacgatgcgtactgggcggcgaaagcgattgtgccggatgcgcgtgttattagcaccgattgcatcgtgccgattagccgtctgggtgaactgatcgagggcgttcaccgtgacattgaagcgagcggtctgcgtgcgccgctgctgggtcacgtgggtgatggcaacttccacaccctgatcattaccgacgataccccggagggtcaccagcaagcgctggacctggatcgtaagatcgtggcgcgtgcgctgagcctgaacggtagctgcagcggcgaacacggtgttggcatgggcaagctggagtttctggaaaccgaacacggcccgggtagcctgagcgttatgcgtgcgctgaaaaacaccatggatccgcatcacatcctgaatccgggcaagctgctgccgccgggtgcggtttataccggttaatga

SEQ ID NO: 7 - DNA, 1571 bp, Artificial Sequence
  Polynucleotide
cttgataaggtccacgtagctgctatagttgcttcaacagaacatattgactatccggtattacccggccctctagaaataattttgtttaactttaagaaggagatatacatatgcaccaccaccaccaccacatgcccgaaccagtaatgacagcttcaagcgcgagcgcgccggatcgtctgcaagcggttctgaaggcgctgcagccggtgatgggtgaacgtatcagcaccgcgccgagcgttcgtgaggaacacagccacggcgaggcgatgaacgcgagcaacctgccggaagcggtggtttttgcggagagcacccaggatgtggcgaccgttctgcgtcactgccacgaatggcgtgtgccggtggttgcgtttggtgcgggcaccagcgttgaaggtcacgtggttccgccggagcaagcgatcagcctggacctgagccgtatgaccggcattgtggatctgaacgcggaggacctggattgccgtgttcaggcgggtatcacccgtcaaaccctgaacgtggaaattcgtgacaccggcctgttctttccggttgatccgggtggcgaggcgaccatcggtggcatgtgcgcgacccgtgcgagcggtaccgcggcggtgcgttacggcaccatgaaggaaaacgttctgggtctgaccgtggttctggcgaccggcgagatcattcgtaccggtggccgcgtgcgtaaaagcagcaccggttatgacctgaccagcctgttcgttggcagcgaaggtaccctgggcatcattaccgaggtgcaactgcgtctgcatggccgtccggacagcgttagcgcggcgatctgccaatttgaaagcctgcacgatgcgattcagaccgcgatggagatcattcaatgcggtatcccgattacccgtgtggaactgatggatagcgttcagatggcggcgagcatccaatacagcggtctgaacgaatatcagccgctgaccaccctgttctttgagtttaccggcagcccggcggcggtgcgtgagcaagttgaaaccaccgaggcgattgcgagcggtaacaacggtctgggctttgcgtgggcggaaagcccggaggaccgtacccgtctgtggaaggcgcgtcacgatgcgtactgggcggcgaaagcgattgtgccggatgcgcgtgttattagcaccgattgcatcgtgccgattagccgtctgggtgaactgatcgagggcgttcaccgtgacattgaagcgagcggtctgcgtgcgccgctgctgggtcacgtgggtgatggcaacttccacaccctgatcattaccgacgataccccggagggtcaccagcaagcgctggacctggatcgtaagatcgtggcgcgtgcgctgagcctgaacggtagctgcagcggcgaacacggtgttggcatgggcaagctggagtttctggaaaccgaacacggcccgggtagcctgagcgttatgcgtgcgctgaaaaacaccatggatccgcatcacatcctgaatccgggcaagctgctgccgccgggtgcggtttataccggttaatga

SEQ ID NO: 8 - DNA, 1572 bp, Artificial Sequence
  Polynucleotide
cctgataaggtccacagtagctgctataattgcttcaacagaacatattgactatccggtattacccggccctctagaaataattttgtttaactttaagaaggagatatacatatgcaccaccaccaccaccacatgcccgaaccagtaatgacagcttcaagcgcgagcgcgccggatcgtctgcaagcggttctgaaggcgctgcagccggtgatgggtgaacgtatcagcaccgcgccgagcgttcgtgaggaacacagccacggcgaggcgatgaacgcgagcaacctgccggaagcggtggtttttgcggagagcacccaggatgtggcgaccgttctgcgtcactgccacgaatggcgtgtgccggtggttgcgtttggtgcgggcaccagcgttgaaggtcacgtggttccgccggagcaagcgatcagcctggacctgagccgtatgaccggcattgtggatctgaacgcggaggacctggattgccgtgttcaggcgggtatcacccgtcaaaccctgaacgtggaaattcgtgacaccggcctgttctttccggttgatccgggtggcgaggcgaccatcggtggcatgtgcgcgacccgtgcgagcggtaccgcggcggtgcgttacggcaccatgaaggaaaacgttctgggtctgaccgtggttctggcgaccggcgagatcattcgtaccggtggccgcgtgcgtaaaagcagcaccggttatgacctgaccagcctgttcgttggcagcgaaggtaccctgggcatcattaccgaggtgcaactgcgtctgcatggccgtccggacagcgttagcgcggcgatctgccaatttgaaagcctgcacgatgcgattcagaccgcgatggagatcattcaatgcggtatcccgattacccgtgtggaactgatggatagcgttcagatggcggcgagcatccaatacagcggtctgaacgaatatcagccgctgaccaccctgttctttgagtttaccggcagcccggcggcggtgcgtgagcaagttgaaaccaccgaggcgattgcgagcggtaacaacggtctgggctttgcgtgggcggaaagcccggaggaccgtacccgtctgtggaaggcgcgtcacgatgcgtactgggcggcgaaagcgattgtgccggatgcgcgtgttattagcaccgattgcatcgtgccgattagccgtctgggtgaactgatcgagggcgttcaccgtgacattgaagcgagcggtctgcgtgcgccgctgctgggtcacgtgggtgatggcaacttccacaccctgatcattaccgacgataccccggagggtcaccagcaagcgctggacctggatcgtaagatcgtggcgcgtgcgctgagcctgaacggtagctgcagcggcgaacacggtgttggcatgggcaagctggagtttctggaaaccgaacacggcccgggtagcctgagcgttatgcgtgcgctgaaaaacaccatggatccgcatcacatcctgaatccgggcaagctgctgccgccgggtgcggtttataccggttaatga

SEQ ID NO: 9 - DNA, 1437 bp, Artificial Sequence
  Polynucleotide
atgcccgaaccagtaatgacagcttcaagcgcgagcgcgccggatcgtctgcaagcggttctgaaggcgctgcagccggtgatgggtgaacgtatcagcaccgcgccgagcgttcgtgaggaacacagccacggcgaggcgatgaacgcgagcaacctgccggaagcggtggtttttgcggagagcacccaggatgtggcgaccgttctgcgtcactgccacgaatggcgtgtgccggtggttgcgtttggtgcgggcaccagcgttgaaggtcacgtggttccgccggagcaagcgatcagcctggacctgagccgtatgaccggcattgtggatctgaacgcggaggacctggattgccgtgttcaggcgggtatcacccgtcaaaccctgaacgtggaaattcgtgacaccggcctgttctttccggttgatccgggtggcgaggcgaccatcggtggcatgtgcgcgacccgtgcgagcggtaccgcggcggtgcgttacggcaccatgaaggaaaacgttctgggtctgaccgtggttctggcgaccggcgagatcattcgtaccggtggccgcgtgcgtaaaagcagcaccggttatgacctgaccagcctgttcgttggcagcgaaggtaccctgggcatcattaccgaggtgcaactgcgtctgcatggccgtccggacagcgttagcgcggcgatctgccaatttgaaagcctgcacgatgcgattcagaccgcgatggagatcattcaatgcggtatcccgattacccgtgtggaactgatggatagcgttcagatggcggcgagcatccaatacagcggtctgaacgaatatcagccgctgaccaccctgttctttgagtttaccggcagcccggcggcggtgcgtgagcaagttgaaaccaccgaggcgattgcgagcggtaacaacggtctgggctttgcgtgggcggaaagcccggaggaccgtacccgtctgtggaaggcgcgtcacgatgcgtactgggcggcgaaagcgattgtgccggatgcgcgtgttattagcaccgattgcatcgtgccgattagccgtctgggtgaactgatcgagggcgttcaccgtgacattgaagcgagcggtctgcgtgcgccgctgctgggtcacgtgggtgatggcaacttccacaccctgatcattaccgacgataccccggagggtcaccagcaagcgctggacctggatcgtaagatcgtggcgcgtgcgctgagcctgaacggtagctgcagcggcgaacacggtgttggcatgggcaagctggagtttctggaaaccgaacacggcccgggtagcctgagcgttatgcgtgcgctgaaaaacaccatggatccgcatcacatcctgaatccgggcaagctgctgccgccgggtgcggtttataccggttaatga